(19) United States
(12) Patent Application Publication
(10) Pub. No.: US 2023/0243850 A1
(43) Pub. Date: Aug. 3, 2023

(54) METHOD FOR ENRICHING EXOSOMES

(71) Applicant: THERAWIS DIAGNOSTICS GMBH, Munchen (DE)

(72) Inventors: Olaf WILHELM, Munchen (DE); Gabriele SCHRICKER, Munchen (DE); Rudolph NAPIERALSKI, Munchen (DE); Percy KNOLLE, Munchen (DE); Bastian HOCHST, Ebenhausen-Schaftlam (DE); Charlotte FLYNN, Hohenkirchen-Siegertsbmnn (DE)

(21) Appl. No.: 17/996,182

(22) PCT Filed: Apr. 16, 2021
(86) PCT No.: PCT/EP2021/059977

§ 371 Date: Oct. 13, 2022

(30) Foreign Application Priority Data
Apr. 17, 2020 (EP) 20170164.6

(51) Int. Cl. G01N 33/68; G01N 33/569; G01N 33/574

(52) U.S. Cl. CPC G01N 33/6893; G01N 33/56983; G01N 33/574

(57) ABSTRACT

The present invention relates to methods for producing an exosome enriched fraction from a sample. The method comprises the steps of contacting a first binding agent that specifically binds to the extra-vesicular part of Rab11 or a second binding agent that specifically binds to the extra-vesicular part of Rab4, or a combination thereof, to the sample under conditions allowing binding of said first binding agent and/or second binding agent to exosomes; and separating exosomes to which the first binding agent and/or the second binding agent is bound from the sample. Further provided are methods for diagnosing cancer and viral diseases, methods for quantifying and/or qualifying tumor-related and virus-related exosomes in a sample, methods for monitoring tumor growth and viral diseases. Also provided is a kit comprising means for carrying out the methods.

### FIELD OF THE INVENTION

The present invention relates to the field of exosomes and in particular to a method for producing an exosome enriched fraction from a sample using binding agents against the extracellular part of Rab11 and/or Rab4. The present invention further pertains to methods for diagnosing cancer and virus infections, methods for quantifying and/or qualifying tumor-related exosomes (oncosomes) and virus-related exosomes (virosomes) in a sample, methods for monitoring tumor growth and infectious diseases, and kits comprising reagents and instructions for carrying out the methods.

### BACKGROUND OF THE INVENTION

Exosomes have first been reported in 1983 when culturing immature red blood cells with labeled transferrin receptors to trace the movement of the transferrin receptors from plasma membranes into the reticulocytes. It was observed that the labeled transferrin receptors were internalized within the reticulocytes, and then repackaged into small vesicles inside them (Harding et al. 1983; Pan et al. 1983). These vesicles were later termed “exosomes” (Johnstone et al. 1989).

Exosomes belong to a large family of membrane vesicles referred to as extracellular vesicles (EVs), which generally include microvesicles (approx. 100-350 nm in diameter), apoptotic blebs (approx. 500-1000 nm in diameter), and exosomes (approx. 30-150 nm in diameter) (Li et al. 2017). Exosomes are thus the smallest type of extracellular microvesicles and are produced in inward budding multivesicular bodies (MVB) resulting in intra-luminal vesicles (ILV). If an MVB fuses with the cell surface (the plasma membrane), these ILVs are released as exosomes by exocytosis into the extracellular media. Exosomes can also be produced by the Golgi apparatus. These exosomes will be released through late endosomes (McAndrews and Kallun. 2019). Exosomes have a characteristic lipid bilayer which has an average thickness of about 5 nm. The lipid components of exosomes include ceramide, cholesterol, sphingolipids, and phosphoglycerides with long and saturated fatty-acyl chains. The outer surface of exosomes is rich in saccharide chains, such as mannose, polylactosamine, alpha-2,6 sialic acid, and N-linked glycans (summarized in Li et al. 2017).

In addition to performing many biological functions, particularly cell-cell communication, cumulative evidence has suggested that several biological entities in exosomes like proteins and microRNAs are closely associated with the pathogenesis of most human malignancies (Li et al. 2017).

As stated above, exosomes are generated in various compartments of the cell, e.g. endosomes and Golgi apparatus. The different processes leading to the generation of exosomes are regulated by Rab GTPases. Among these GTPases, Rab4 seems to be involved in the recycling of exosomes from early endosomes, and Rab11 regulates the slow transport from perinuclear recycling endosome compartment. Rab11 is further proposed to regulate the transport of microvesicle endosomes to the plasma membrane and thus to exosome release (Blanc and Vidal 2018). It has been shown that overexpression of Rab11 stimulates the exosome release in K562 cells, and the inhibition of Rab11 function decreases exosome release (Savina et al. 1997).

Secretion of exosomes occurs from normal (thrombocytes, immune cells, etc.) and tumor cells. They can carry DNA, RNA, microRNA, proteins, and lipids. Exosome-associated proteins used as biomarkers today include tetraspanins (CD9, CD63, CD81), immune regulation molecules (HLA-G, MHCI/II), membrane transport and fusion proteins (Rab5). The molecular composition of exosomes is assumed to reflect the (patho-) physiological changes in their cell or tissue of origin (Jia et al. 2017).

Viruses enter cells through the endocytic pathway. Viruses that enter through endocytosis can hijack and use exosomal pathways for their own benefit. Exosomes have several characteristics that are like some viruses. These characteristics include biogenesis, uptake by cells, and intercellular transfer of functional RNAs, mRNAs, and proteins. Virus-infected cells have been shown to secrete exosomes that vary from their viral counterparts but may comprise of viral RNAs and viral proteins (Crenshaw et al. 2018). Such exosomes are referred to as “virosomes”. Thus, identification of exosomes released from cells upon viral infection (virosomes), and identification of viral proteins comprised in said virosomes will allow diagnosing virus infection with high sensitivity and independent of DNA or RNA analysis.

However, the isolation, enrichment and detection of exosomes has proven to be complicated (van der Pol et al 2012; Thind et al. 2016; Jia et al. 2016). Due to the complexity of body fluids, physical separation of exosomes from cells and similar-sized particles turned out to be challenging. Methods have been applied to isolate exosomes such as immune-capturing with antibodies, ultracentrifugation, and precipitation with PEG6000, water deprivation etc. The isolation of exosomes using differential ultracentrifugation was found to result in co-isolation of proteins and other contaminants and incomplete separation of vesicles from lipoproteins. Combining ultracentrifugation with micro-filtration or a gradient was suggested to improve purity (Tauro et al. 2012; van Deun et al. 2014). Further, a single step isolation of extracellular vesicles by size-exclusion chromatography has been demonstrated to provide greater efficiency for recovering intact vesicles over centrifugation (Boing et al. 2014), although a size-based technique alone will not be able to distinguish exosomes from other vesicle types. In addition, when applying these conventional methods, a mixed population of exosomes of different intracellular origin and further vesicles are generally co-purified.

Commercially available exosome enrichment and/or isolation kits include the “Total Exosome Isolation Reagent” from Invitrogen (distributed by ThermoFisher Scientific), the “Exo-spin kit” from Cell Guidance Systems, and the “exoEasy Maxi Kit” from Qiagen. However, the present inventors show that none of the methods or kits known in the art satisfyingly enriches a significant number of exosomes of a sample, but rather small numbers or a fraction or subpopulation thereof only (see example 1 below).

In order to develop, for example, a sensitive tumor-specific exosome test or virus-specific exosome test, it is, however, necessary to purify all or at least the majority of exosomes comprised in a sample, which are then likely to comprise in addition to exosomes originating from normal cells also those originating from tumor and/or virus infected cells. In this regard it is particularly desirable to isolate and enrich tumor- and virus-specific exosomes (oncosomes, and virosomes) with high purity and quality for subsequent analysis in clinical diagnostics, e.g. detection of tumor biomarker or viral proteins. By using for example, the blood of a healthy patient, of a patient suspected of having a tumor disease, or of a patient having a tumor disease as test sample, exosome characterization within the blood or fraction thereof, e.g. serum, may serve as “liquid biopsy”, allowing an alternative, less invasive sampling for initial diagnosis, characterization and/or staging of the tumor disease as well as prognosis, which can be applied even if the tumor is not directly accessible or if repeat biopsies is not feasible. Thus, such an analytical method will enable cancer screening, therapy monitoring, as well as monitoring/detecting disease progression and/or recurrence. Similarly, patient samples, e.g. blood or fractions thereof, can be screened for viral proteins to detect or monitor viral infection.

There is, thus, a long felt need in the field for a rapid, reliable, specific and sensitive method for purifying and isolating exosomes. Because exosomes derive from different intracellular origins, it is essential to isolate various exosome subpopulations. Surprisingly, Rab11 and Rab4 which are responsible for exosome regulation and trafficking, are also components of the exosomal membrane, and thus, can be utilized to identify and isolate exosomes from different intracellular sources. Significantly, exosomes which are released from virus infected cells contain Rab11, which allows the identification/isolation of virosomes and the application of secondary binding molecules directed against one or more virus specific protein for diagnosing and monitoring viral diseases. It is also envisioned that viral nucleic acids, RNA or DNA, may be detected in exosomes.

### SUMMARY OF THE INVENTION

In a first aspect, the present invention provides a method for producing an exosome enriched fraction from a sample, the method comprising the steps of contacting a first binding agent that specifically binds to the extracellular part of Rab11 or a second binding agent that specifically binds to the extracellular part of Rab4, or a combination thereof, to the sample under conditions allowing binding of said first binding agent and/or second binding agent to exosomes; and separating exosomes to which the first binding agent and/or the second binding agent is bound from the sample.

According to one embodiment, the first binding agent comprises a first label and/or the second binding agent comprises a second label. Alternatively, the first binding agent and/or the second binding agent is bound by a third binding agent specifically binding to the first binding agent and/or the second binding agent, wherein the third binding agent comprises a third label. Alternatively, the first binding agent and/or second binding agent is covalently or non-covalently bound on a solid surface.

According to one embodiment, the first, the second and the third label is independently selected from the group consisting of an enzyme label, a fluorescence label, a radioactive label, a magnetic label, a peptide or protein label, or a quantum dot.

According to yet another embodiment, the step of separating the exosomes comprises the step of detecting the first and/or second antigen binding agent. Alternatively, the step of separating the exosomes comprises the step of detecting the third antigen binding agent.

According to one embodiment, the sample is a body fluid. Preferably, the body fluid is selected from the group consisting of plasma, ascites, cerebral fluid, bone marrow, urine, faeces or bronco-alveolar washing.

According to yet another embodiment, the first binding agent binds to the extracellular part of Rab11A and the second binding agent binds to the extracellular part of Rab4A.

According to one embodiment, the method further comprises prior the step of contacting the first and/or second binding agent with the sample the steps of suspending or solubilizing the sample, and enriching exosomes from the sample based on size and/or density.

According to another embodiment, the step of separating the exosomes comprises flow cytometry, magnetic or microbead separation or chromatography.

According to a further aspect, the present invention provides a method for diagnosing cancer. The method comprises producing an exosome enriched fraction by the method of the present invention, and detecting within the exosome enriched faction exosomes presenting a cancer antigen. Preferably, said cancer antigen is GPER-1. More preferably, the detection step comprises detecting the GPER-1 presenting exosomes with an anti-GPER-1 antibody.

According to a preferred embodiment, the cancer is breast cancer.

According to a further aspect, the present invention provides a method for diagnosing a virus disease. The method comprises producing an exosome enriched fraction by the method of the present invention, and detecting in the exosome enriched fraction exosomes presenting or carrying a virus antigen. Alternatively or additionally, viral nucleic acid, i.e. DNA or RNA may be detected in the exosome enriched fraction.

According to a further aspect, the present invention provides a method for quantifying and/or qualifying tumor-related exosomes in a sample. The method comprises the steps of producing an exosome enriched fraction by the method of the present invention, and detecting tumor-related exosomes in the exosome enriched fraction of step a) with at least one binding agent specifically binding to a tumor antigen. The tumor antigen is preferably selected from the group consisting of GPER-1, CD247, and phosphatidylserine.

According to a yet another aspect, the present invention provides a method for monitoring tumor growth. The method comprises the step of periodically quantifying the number of tumor related exosomes in a sample with the method of the present invention. An increase in the number of tumor related exosomes between two quantifications thereby indicates tumor growth.

According to a yet another aspect, the present invention provides a method for monitoring a virus disease. The method comprises the step of periodically quantifying the number of virus related exosomes in a sample with the method of the present invention. The method may further comprise the step of detecting within the exosome enriched faction obtained those exosomes presenting a viral antigen. Preferably the viral antigen is a virus surface protein, more preferably a spike protein, most preferably a spike protein of the SARS-CoV-2 or Sars-CoV-1 virus.

According to an embodiment, the sample used in any of the methods of the present invention is obtained from a subject known or suspected to suffer from a disease, and the method further comprises the step of comparing the quantity of exosomes in the sample of the subject known or suspected to suffer from a disease with the quantity of similar exosomes known to be present in a sample of a healthy subject. Thereby, an increase in the quantity of exosomes in the sample of the subject known or suspected to suffer from a disease is indicative of the presence or stage of the disease, preferably indicative of the presence or stage cancer. Comparing the quantity of exosomes preferably comprises applying CD mapping and tSNE analysis.

According to another embodiment, the method of the present invention further comprises analyzing surface markers and/or the content of the exosomes. Preferably, the method comprises analyzing proteins, peptides, microRNA, DNA, and/or RNA. More preferably, said analyzing the content of the exosomes comprises DNA mutation analysis, RNA expression, DNA methylation quantification and/or protein expression.

According to a preferred embodiment, the antigen binding agent is an antibody.

According to a further aspect, the present invention provides a kit for performing the method of the present invention. The kit comprises a first binding agent that specifically binds to the extracellular part of Rab11 or a second binding agent that specifically binds to the extracellular part of Rab4, or a combination thereof, and instructions for using the first and/or the second binding agent for binding of said first and/or second binding agent to exosomes in a sample.

According to another aspect, the present invention provides a method of diagnosing cancer or virus diseases in a subject, the method comprising the steps of a) obtaining a sample from a person known to or suspected of having cancer or virus infection; b) producing an exosome enriched fraction from the sample, comprising: i) contacting a first binding agent that specifically binds to the extracellular part of Rab11 or a second binding agent that specifically binds to the extracellular part of Rab4, or a combination thereof, to the sample under conditions allowing binding of said first binding agent and/or second binding agent to exosomes; and ii) separating exosomes to which the first binding agent and/or the second binding agent is bound from the sample; and c) detecting tumor-related or virus-related exosomes in the exosome enriched fraction of step a) with at least one binding agent specifically binding to a tumor or virus antigen.

According to an embodiment, the first binding agent comprises a first label and/or the second binding agent comprises a second label. Alternatively, the first binding agent and/or the second binding agent is bound by a third binding agent specifically binding to the first binding agent and/or the second binding agent, wherein the third binding agent comprises a third label. Alternatively, the first binding agent and/or second binding agent is covalently or non-covalently bound on a solid surface.

According to one embodiment, the first, second and third label is independently selected from the group consisting of an enzyme label, a fluorescence label, a radioactive label, a magnetic label, a peptide or protein label, or a quantum dot.

According to an embodiment, the sample is a body fluid. Preferably, the body fluid is selected from the group consisting of plasma, ascites, cerebral fluid, bone marrow, urine, faeces or bronco-alveolar washing.

According to an embodiment, the first binding agent binds to the extracellular part of Rab11A and the second binding agent binds to the extracellular part of Rab4A.

According to an embodiment, the method further comprises prior to step b) the steps of suspending or solubilizing the sample, and enriching exosomes from the sample based on size and/or density.

According to an embodiment, the method further comprises in step ii) preforming flow cytometry or chromatography.

According to an embodiment, the tumor antigen is selected from the group consisting of GPER-1, CD247, or phosphatidylserine.

According to an embodiment, the virus antigen is a virus surface protein, preferably a spike protein, more preferably a spike protein of the SARS-CoV-2 or Sars-CoV-1 virus.

According to an embodiment, the antigen binding agent is an antibody.

Further aspects and embodiments of the present invention are derivable from the following detailed description and the examples.

### FIGURES

FIG. 1: Exosome enrichment with various available methods. Exosome containing fractions were isolated from plasma of healthy donors via a Pancoll gradient. Subsequently, FACS analysis was performed with exosome markers Rab5, CD41, CD81, HLA-ABC. FIGS. 1A and 1B show FACS results. FIG. 1A from left to right: plasma sample unstained; plasma sample; ultra-centrifugation sample. FIG. 1B from left to right: sample from using Total Exosome Isolation; sample from using ExoSpin; sample using PEG6000; sample using ExoEasy. Gates in the graphs depict exosome fractions.

FIG. 2: Exosome enrichment with ultra-centrifugation or PEG6000 purification. Gates in the graphs depict the exosome fractions. FACS results for from left to right: plasma sample; ultracentrifugation sample, PEG6000 sample.

FIG. 3: (A) Identification of Rab4+, Rab11+ and Rab5+ exosomes from plasma and cancer cell line supernatants. Top row plasma samples, bottom row cancer cell line samples. FACS results for from left to right: Forward Scatter (FSC)/Side Scatter (SSC); staining with Rab5; staining with Rab4; staining with Rab11. (B) Detection of CD340 (HER2) on Rab11 positive exosomes by means of flow cytometry (left panel); control antibody with Rab11 positive exosomes (right panel).

FIG. 4: Exosome enrichment with ultra-centrifugation or PEG6000 purification and using Rab4 staining. Gates in the graphs depict the exosome fractions. FACS results for from left to right: plasma sample; ultracentrifugation sample, PEG6000 sample.

FIG. 5: FACS results for identification of Rab11+ exosomes in a plasma sample, an ultra-centrifugation sample and in a PEG6000 sample. Rab11 staining results in higher exosome quantity than using Rab5 or Rab4 staining in comparable samples.

FIG. 6: Improving the available methods by Rab11 staining. FIG. 6A, FACS results for from left to right: plasma sample untreated; plasma sample; ultra-centrifugation sample.

FIG. 6B, FACS results for from left to right: sample from using Total Exosome Isolation; sample from using ExoSpin; sample using PEG6000; sample using ExoEasy. Gates in the graphs depict exosome fractions. Using Rab11 staining significantly improves exosome quantity in all samples tested (compare with example 1; FIGS. 1A and 1B).

FIG. 7: (A) Rab11 positive exosome population contains Rab4 and Rab5 positive subpopulations, but not vice versa. (B) Yield determination using different anticoagulants (left panel) and using different storage conditions for the plasma sample (right panel). Rab11 purification demonstrates the highest yields under all conditions tested.

FIG. 8: Surface mapping of exosomes purified from plasma samples of healthy volunteers.

FIG. 9: Surface mapping of exosomes purified from cancer samples.

FIGS. 10 to 14: tSNE analysis of exosomes from healthy volunteers.

FIG. 15: FACS analysis of virosome enrichment using Rab11 antibody. Left: virosome enrichment using Rab11; right: control.

FIG. 16: Flow cytometric detection of small EVs and multiparametric analysis through fluorochrome-labeled antibodies showing expression of from left to right Rab4, Rab5 and Rab11 in EVs.

FIG. 17: Fluorescence flow cytometry: upper panel isolation with Rab11 antibody coupled to magnetic beads from human plasma, lower panel exosomes purified from the same plasma sample using Qiagen's ExoEasy Isolation kit. Rab11 is superior in purifying exosomes from human plasma compared to an isolation using Qiagen ExoEasy Purification kit.

FIG. 18: Detection of SARS-CoV-2 spike antigen on exosomes in blood of Covid-19 patients. Results show that Rab11 positive exosomes carrying SARS-CoV-2 spike protein can be utilized for diagnosing a viral disease.

FIG. 19: Stochastic neighborhood analysis of flow cytometric results from phenotypic profiling of Rab11 positive exosomes from human plasma counterstained with the indicated marker.

FIG. 20: Exosome staining in samples from plasma of healthy volunteers and supernatant of a mixture of breast cancer cell line (BT474, SKBR3, MCF7, OVCAR2, OVMZ6) with fluorescently labelled antibodies against Rab11, CD9, CD81, HLA-ABC and CD63. Donor 1, 2, 3=plasma samples of three different healthy volunteers; ZK-SN=cell culture supernatant of breast cancer cell lines.

### DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolb′, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

### DETAILED DESCRIPTION OF THE INVENTION

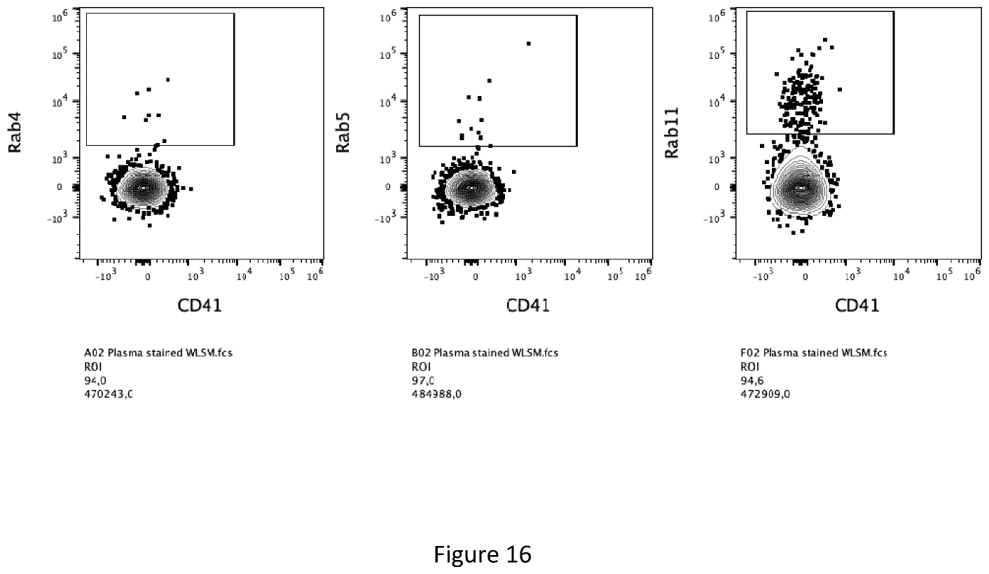

The present inventors surprisingly found that Rab11 and/or Rab4 are particularly good markers of exosomes, i.e. they are consistently present in detectable amounts in the majority of exosomes but to a much lesser degree in other extracellular vesicles (EVs). Specifically, the inventors found that current markers for EVs such as CD9, CD63 or CD81 do not detect the entire population of extracellular vesicles in the blood of humans. Further, CD9, CD63 and CD81 also detected extracellular vesicles derived from platelets. In addition to expression of Rab4 and Rab5, another member of the Rab GPTase, Rab11, was identified as marker for EVs that identified a much larger population of EVs than others (FIG. 16). Noteworthy, Rab11 capturing delivers the highest yield in comparison to Rab5 or other markers. Without wishing to be bound by any theory, it is assumed that immunocapturing with Rab11 isolates various subpopulations of exosomes since Rab11 is involved in the formation and release of exosomes from recycling and late endosomes as well as from the Golgi apparatus. Thus, detection of vesicles comprising Rab11 and/or Rab4 on its surface allows the isolation of a larger number of exosomes from a given sample comprising EVs than prior art methods. Without wishing to be bound by any theory, it is assumed that Rab4 and Rab11 are not only regulatory elements, but they are also trapped or localized in the exosome surface and, thus, are useful targets in a purification method. By targeting Rab11, exosomes from different origins within the cell compartments can be identified, e.g. from recycling endosomes, late endosomes and Golgi apparatus. The present inventors have further surprisingly found that capturing with Rab 11 detects CD41 negative EVs but not CD41 positive microvesicles. Thus, using Rab 11 allows isolating pure exosomes without contamination with platelet-derived microvesicles. In the present invention it is further shown that exosomes from healthy volunteers differ from those derived from subjects known or suspected to suffer from a disease, allowing enrichment and purification of such specific exosomes and their subsequent analysis. Therefore, according to a preferred embodiment, the method of the present invention comprises the step of contacting a first binding agent that specifically binds to the extracellular part of Rab11 to the sample under conditions allowing binding of said first binding agent to exosomes.

Accordingly, the present invention provides methods for producing an exosome enriched fraction from a sample, methods for diagnosing cancer, methods for quantifying and/or qualifying tumor-related exosomes in a sample, methods for monitoring tumor growth, and kits comprising reagents and instructions for carrying out the methods.

The term “subject” as used herein refers to an individual, such as a human, anon-human primate (e.g. chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. In a particular meaning, the subject is a mammal. In a preferred meaning, the subject is a human. The subject can be a healthy subject or a subject suffering from or suspected of having one or more diseases. A subject suffering from or suspected of having one or more diseases is also referred to as a patient.

The term “sample” as used herein refers to biological material obtained from a subject. A sample can be obtained from any suitable tissue or biological fluid such as nipple aspirate, blood, serum, plasma, ascites, cerebral fluid, bone marrow, urine, faeces or bronco-alveolar washing. Preferably, said biological sample is provided in a state selected from the group consisting of natural, frozen, lyophilized, preserved, embedded, and all possible combinations thereof. Methods for deriving samples from a subject are well known to those skilled in the art. The methods of the present invention are preferably carried out with samples obtained from a subject suffering from or suspected of having one or more diseases.

Cell sorting in general describes the process of purifying or enriching cell populations based on the presence or absence of specific physical characteristics. In flow cytometers with sorting capabilities, the instrument detects cells using parameters such as cell size, morphology, and protein expression. Droplet technology is then used to sort cells and recover the subsets. This principle can also be applied for purifying or enriching e.g. cell components such as exosomes. The term “FACS” used herein refers to fluorescence activated cell sorting.

The term “Rab” as used herein refers to Rab GTPases which are described in the art as regulators in the generation of exosomes. The Rab11 subfamily is described as including Rab11a, Rab11 b and Rab11c (Balnc and Vidal, 2018). Rab11 and Rab4 are used as targets in the present invention for identifying exosomes, allowing their subsequent enrichment or isolation. Since Rab11 and Rab4 are located on the surface of the exosome, the binding agents that specifically bind to Rab11 and/or Rab4 bind to that part of Rab11 and/or Rab4, that is accessible from the outside of the exosome. This accessible part is herein described as the “extra-vesicular part” of Rab11 and/or Rab4.

The term “agent” as used herein denotes a chemical compound, a mixture of chemical compounds, a biological macromolecule, or an extract made from biological materials.

The term “binding agent” as used herein pertains to any agent capable of specifically and/or selectively binding to a specific biological structure. In the context of the present invention, the term ‘binding agent specifically binding to the extra-vesicular part of Rab11 or Rab4’ is used to denote agents that specifically and/or selectively bind Rab11 or Rab4, in particular to their extra-vesicular part. Since Rab11 and Rab4 are proteins, said binding agent specifically and/or selectively binds to a part of the amino acid structure of said Rab GTPases. Such binding agent can therefore be an antigen binding agent or molecule such as an antibody or a functional binding fragment thereof capable of binding to said Rab GTPases. Certain binding proteins described herein are antibodies or are derived from antibodies. In certain embodiments, the polypeptide structure of the antigen binding proteins is based on antibodies, including, but not limited to, monoclonal antibodies, bispecific antibodies, minibodies, nanobodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the binding agent comprises or consists of avimers (tightly binding peptide). The binding agent can comprise all or part of a variable fragment (Fᵥ) of an antibody or of the variable α- and β-chains or γ- and δ-chains of a T cell receptor (TCR). The binding agent can also be a fusion molecule comprising parts of a TCR and parts of an Fᵥ, as long as it allows its specific and/or selective binding to its target. The binding agent can be a single valent binding agent, bivalent binding agent or a multi-valent binding agent, having two or more binding sites. The above description of a binding agent applies likewise to binding agents described herein as not binding to Rab11 or Rab4 but for example binding to a further binding agent.

If the binding agent is an antigen binding agent, it “specifically binds” a target antigen when the dissociation constant (K_{d}) is ≤10⁻⁷ M. The binding agent specifically binds its antigen with “high affinity” when the K_{d} is ≤5×10⁻⁹ M, and with “very high affinity” when the K_{d} is ≤5×10⁻¹⁰ M.

If the binding agent is an antigen binding agent, it is “selective” when it binds to one target more tightly than it binds to a second target.

As used herein, the terms “label” or “labeled” refer to incorporation of a detectable marker, e.g., by incorporation of a respectively labeled amino acid or attachment to a polypeptide of the binding agent. The term also encompasses dyes and stains.

The term “methylation” or “DNA methylation” as used herein refers to a biochemical process involving the addition of a methyl group to the cytosine or adenine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site.

The term “antigen” as used herein refers to a molecule or a portion of a molecule capable of being bound by a selective binding agent, such as an antigen binding protein (including, e.g., an antibody or immunological functional fragment thereof). Antigens can possess one or more epitopes that are capable of interacting with different antigen binding proteins.

The term “epitope” as used herein includes any determinant capable being bound by an antigen binding protein, such as an antibody or to a T cell receptor. An epitope is a region of an antigen that is bound by an antigen binding protein that targets that antigen, and when the antigen is a protein, includes specific amino acids that directly contact the antigen binding protein.

The term “flow cytometry” as used herein refers to technique used to detect and measure physical and chemical characteristics of a population of cells or particles. Flow cytometers that can be used in this context are commercially available and well established in the field of cell biology.

The term “flow chromatography” as used herein refers to a chromatography method to isolate single compound or group of compounds from a mixture. Chromatography separates substances based on differential adsorption of compounds to the adsorbent and makes use of a column carrying the adsorbent. The mixture containing the compounds moves through the column at different rates, allowing the mixture to be separated into different fractions.

Exosomes “presenting” or “carrying” a molecule such as a peptide, protein or antigen as referred herein means that the molecule is at least partially accessible from the outside of the exosome. For example, a respective molecule may be located on the exosome's outer membrane or at least a portion of said molecule may be located on the exosome's outer membrane. A respective presented or carried molecule may also spun or extend through the exosome's membrane.

The term “extra-vesicular part” as used herein denotes the part of a molecule such as a peptide or protein present in or on an exosome that is accessible from the outside of the exosome.

### Methods of the Invention

There is a long felt need in the art for a rapid, reliable, specific and sensitive method for enriching exosomes, allowing purification and isolation thereof with subsequent analysis of the exosome contents such as microRNA, DNA, DNA methylation and RNA. This need has now been met by the methods according to the present invention.

According to a first aspect, the present invention provides a method for producing an exosome enriched fraction from a sample. According to one embodiment, the sample is a body fluid. Preferably, the body fluid is selected from the group consisting of plasma, ascites, cerebral fluid, bone marrow, urine, faeces or bronco-alveolar washing. Obtaining such sample is well known in the art and is not limited to any particular method. Depending on the sample type or its state, the sample can be suspended or solubilized before being used in the method of the present invention. According to a preferred embodiment, the sample is an unfractionated sample of blood.

The subject from which the sample is derived or obtained is preferably a mammal, most preferably a human. Thus, according to a particularly preferred embodiment, the sample is an unfractionated sample of human blood.

The size of the sample is not particularly limited but preferably includes low amounts for keeping the distress of the subject to a minimum. If the sample is a liquid sample, amounts of 10 ml or below can be used, preferably not more than 5 ml, 4 ml, 3 ml, 2 ml, 1 ml, or 0.5 ml. According to a most preferred embodiment, the sample size is not more than 2 ml. By taking a low amount of a liquid sample, the methods of the present invention can be included in a so called liquid biopsy as described e.g. in Hench et al., 2018.

The method according to the present invention comprises the steps of: i) contacting a first binding agent that specifically binds to the extra-vesicular part of Rab11 or a second binding agent that specifically binds to the extra-vesicular part of Rab4, or a combination thereof, with the sample under conditions allowing binding of said first binding agent and/or second binding agent to exosomes; and ii) separating exosomes to which the first binding agent and/or the second binding agent is bound from the sample. The terms first and second binding agents do not define the exact number of binding agents. Rather, these binding agents are defined by their functionality of binding to either the extra-vesicular part of Rab11 or to the extra-vesicular part of Rab4. Thus, each binding agent may comprise one or more different types of binding agents, each such group being united by the common function of its members to specifically bind to either to Rab11 or Rab4. It is emphasized that such binding of a group of binding agents does not require binding to exactly the same antigen on Rab4 or Rab11. Within each group of binding agents, different subgroups may bind to a different antigen of the target protein, as long as all subgroups within the same group bind to the same target, i.e. Rab4 or Rab11. The method of the present invention may thus comprise contacting one or more types of binding agents specifically binding to the extra-vesicular part of Rab11 with the sample. Alternatively, the method may comprise contacting one or more types of binding agents specifically binding to the extra-vesicular part of Rab4 with the sample. Alternatively, the method may comprise contacting one or more types of binding agents specifically binding to the extra-vesicular part of Rab11, and one or more types of binding agents specifically binding to the extra-vesicular part of Rab4 with the sample. According to a particularly preferred embodiment of the invention, the method comprises contacting a first binding agent that specifically binds to the extra-vesicular part of Rab11 with the sample.

According to one embodiment of the present invention, the first binding agent binds to the extra-vesicular part of Rab11, preferably to the extra-vesicular part of Rab11A, and the second binding agent binds to the extra-vesicular part of Rab4, preferably to the extra-vesicular part of Rab4A.

The conditions allowing binding of the first and/or the second binding agent to the sample depend in particular on the type of binding agent used and on the type of sample. The conditions can be easily determined by a person skilled in the field.

For detecting the binding agent bound to the extra-vesicular part of Rab11 and/or Rab4, the first binding agent may comprise a first label and/or the second binding agent may comprise a second label. Alternatively, after binding to Rab11 and/or Rab4, the first binding agent and/or the second binding agent can be subsequently bound by a third binding agent specifically binding to the first binding agent and/or the second binding agent. In such cases, the third binding agent comprises a third label. Such third binding agent can also be a group of different binding agents—in line with the definition of the first and second binding agent above—which different binding agents are united by their common function of binding to the first or the second binding agent.

The first, second and third label can be independently selected from the group consisting of an enzymatic label, a fluorescence label, a radioactive label such as isotopes or radionuclides, a magnetic label such as magnetic beads, and a peptide or protein label. Examples of peptide and protein labels include but are not limited to biotin and avidin/streptavidin. Commonly used enzymatic labels, fluorescence labels, radioactive labels, and magnetic labels can be used in the context of the present invention without any specific limitation thereto. Respective examples include but are not limited to ³H, ¹⁴C, ¹⁵N, ³⁵S, ⁹⁰Y, ⁹⁹Tc, ¹¹¹In, ¹²⁵I, ¹³¹I for radioactive labels; FITC, rhodamine, lanthanide phosphors for fluorescent labels; horseradish peroxidase, β-galactosidase, luciferase, and alkaline phosphatase for enzymatic labels; and chemiluminescent labels. Quantum dots can also be used as label. Quantum dots are semiconductor nanocrystals that have broad excitation spectra, narrow emission spectra, tunable emission peaks, long fluorescence lifetimes, negligible photobleaching, and ability to be conjugated to proteins and are exemplary described in Barroso, 2011. There is also no specific limitation regarding the use of quantum dots in the present invention.

In certain embodiments, labels are attached by spacer arms of various lengths to reduce potential steric hindrance. Such spacers can be, for example, chemical spacers or amino acid spacers.

In an alternative embodiment of the present invention, the first binding agent and/or the second binding agent is covalently or non-covalently bound on a solid surface, as in an array setting. Such array construct may be used for binding the exosomes to respective probes comprising the binding agents. Accordingly, the present invention also provides an array comprising the first and/or second binding agents, and the use of such array for enriching or isolating exosomes from a sample. If bound to an array, the exosomes can be released after washing the array to remove any contaminants.

According to an embodiment of the present invention, step ii) of the method for producing an exosome enriched fraction from a sample comprises the step of detecting the first and/or second antigen binding agent. Alternatively, step ii) of the method comprises the step of detecting the third antigen binding agent. Detecting the first, second or third binding agent can be performed by any method known in the art. If a label is attached to the first, second and/or third binding agent, it is envisioned to detect the label. For example, biotin moieties can be detected by marked avidin (e.g., streptavidin containing a fluorescent marker or enzymatic activity that can be detected by optical or colorimetric methods), and fluorescent labels can be detected by exciting the fluorophore and detecting the emitted fluorescence of the fluorophore. Respective detection methods are well known to the person of skill in the art.

According to a further embodiment, step ii) of the method of the present invention comprises applying or using flow cytometry or chromatography. A preferred method for identifying the exosomes is the use of fluorescence activated cell sorting (FACS), which method is well established in the field and can be used not only for separating cells but also for separating vesicles such as exosomes. Alternatively, the exosomes can be identified on a column or on an array such as a chip array, in which the binding agents are coupled to a surface and serve as probes for capturing the exosomes.

According to a preferred embodiment, exosomes are enriched in the sample based e.g. on size and/or density before the sample is contacted with the first and/or second binding agent. Such enrichment of exosomes can be performed e.g. by density centrifugation or ultra-centrifugation using e.g. a Pancoll gradient (PAN-Biotech GmbH). Other suitable gradients include but are not limited to Ficoll and Ficoll-Paque (both GE Healthcare), and Biocoll (Biochrom GmbH). Methods of performing ultra-centrifugation are well known to the person of skill in the art and are disclosed for example in Li et al., 2017.

In order to develop, for example, a tumor-specific exosome test, it is necessary to purify all or at least the majority of exosomes derived from normal and tumor cells, and to isolate and enrich tumor-specific exosomes (oncosomes) with high purity and quality for subsequent tumor biomarker analysis in clinical diagnostics. By using for example the blood from cancer patients as test sample, exosome characterization may serve as “liquid biopsy”, allowing an alternative, less invading sampling, which can even be applied if tumor tissue is not directly accessible. A respective analytical method further enables to screen for cancer, to monitor therapy, disease progression and recurrence. Accordingly, the present invention provides a method for diagnosing cancer. The method comprises the steps of a) producing an exosome enriched fraction by the method for producing an exosome enriched fraction of the present invention, and b) detecting within the exosome enriched fraction exosomes presenting a cancer antigen, preferably wherein the cancer antigen is GPER-1 (G protein-coupled estrogen receptor 1). A significantly increased number of exosomes presenting the cancer antigen and preferably GPER-1 compared with a reference sample of a healthy person is indicative of the person being at risk or suffering from cancer. Preferably, GPER-1 is detected by using a binding agent specifically and/or selectively binds GPER-1. Preferably, said binding agent is an antigen-binding agent, and more preferably an antibody against GPER-1. According to one embodiment, the method of diagnosing cancer is used for diagnosing breast cancer. Alternatively or in addition to detecting GPER-1, also GPER-5, CD247 (T cell surface glycoprotein CD3 zeta chain; Cluster of Differentiation 247) and/or phophatidylserine can be used for detecting such oncosomes.

According to a further aspect, the present invention provides a method for quantifying and/or qualifying tumor-related exosomes in a sample. The method comprises the steps of a) producing an exosome enriched fraction by the method for producing an exosome enriched fraction of the present invention, and b) detecting tumor-related exosomes in the exosome enriched fraction of step a) with at least one binding agent specifically binding to a tumor antigen. Such tumor related exosomes (or oncosomes) can be identified by comparing the exosome population of a healthy person with the exosome population of a person suspected of having cancer or already known to suffer from cancer. The tumor antigen can be selected from any tumor antigen known in the art. According to a preferred embodiment, the tumor antigen is selected from the group consisting of GPER-1, GPER-5, CD247, and phophatidylserine. Further exosome surface markers associated with cancer that can be analyzed according to the present invention are CD49b, CD90, CD274 and CD202b.

According to a further aspect, the present invention provides a method for monitoring tumor growth. The method comprising the steps of periodically quantifying the number of tumor related exosomes in a sample with the method of quantifying and/or qualifying tumor-related exosomes in a sample of the present invention. An increase in the number of tumor related exosomes between two quantifications is then indicative of tumor growth.

According to a further aspect, the present invention provides a method for diagnosing a virus diseases. The method comprises producing an exosome enriched fraction by the method of the present invention. Preferably, the method further comprises detecting within the exosome enriched faction exosomes presenting a virus antigen. The present invention further provides a method for monitoring a virus disease. The method comprises the step of periodically quantifying the number of virus related exosomes in a sample with the method of the present invention. The presence of virus related exosomes containing virus derived proteins allows diagnosing and monitoring of the virus disease. The virus derived protein preferably is a virus antigen. According to an embodiment, the virus antigen is a virus surface protein. The virus surface protein is preferably a spike protein, more preferably a spike protein of Riboviria, most preferably of the SARS-CoV-2 or Sars-CoV-1 virus. The virus antigen can be also a subunit of the spike protein e.g. subunit 1 or 2, or peptides derived thereof. Alternatively, the virus protein can be associated with any other virus infection such as for example Influenza A/B, West-Nile-, Zika-, Dengue- or Ebola-virus infections.

The sample used in the methods of the invention can be obtained from a subject known or suspected to suffer from a disease. Any of the methods described herein may further comprise the step of comparing the quantity of exosomes, preferably of disease related exosomes, in the sample of the subject known or suspected to suffer from a disease with the quantity of similar exosomes known to be present in a sample of a healthy subject. An increase in the quantity of the exosomes in the sample of the subject known or suspected to suffer from a disease is then indicative of the presence or stage of the disease. The term “disease related exosomes” is intended to refer to exosomes containing peptides associated with a disease such as viral peptides and antigens as well as cancer peptides and antigens, e.g. as described herein. A disease to be detected or monitored according to the invention is preferably cancer. In such methods, comparing the quantity of exosomes may comprise applying CD mapping and t-SNE analysis. CD mapping includes identifying expression of different Cluster of Differentiation (CD) proteins on the enriched or isolated exosomes and comparing the CD protein expression of one sample with the CD protein expression of another sample. The CD expression profile can then be combined in form of a map. T-distributed Stochastic Neighbor Embedding (t-SNE) is a machine learning algorithm for visualization. It is a nonlinear dimensionality reduction technique well-suited for embedding high-dimensional data for visualization in a low-dimensional space of two or three dimensions. Specifically, it models each high-dimensional object by a two- or three-dimensional point in such a way that similar objects are modeled by nearby points and dissimilar objects are modeled by distant points with high probability. The technique of t-SNE is well known to the person of skill in the art. In the present example 10 it is used to visualize high-level representations learned by an artificial neural network and thus for stochastically confirming the results obtained in the experimental examples. The t-SNE technique may thus be used for providing a further verification process.

Additional analysis of markers such as proteins, peptides and/or antigens in addition to those associated with a disease (such as viral or cancer peptides and/or antigens) may allow localizing the disease to a specific organ or tissue. For example, a disease related exosome may additionally carry or comprise one or more markers for a specific organ or tissue such as cardiac troponin for the heart. Thus, additionally identifying in a population of disease related exosomes organ or tissue specific markers or markers associated with a group of tissues or organs allows associating disease related exosomes to a tissue or an organ, thereby associating the disease to the tissue or organ. Therefore, for further investigating the enriched or isolated exosome populations of a sample, further surface markers and/or the content of the exosomes can be analyzed. Such further surface markers include but are not limited to amyloid-beta, 14-3-3 protein, Actin, ADAM10, Alix, alpha-Enolase, alpha-Synclein, Aminopeptidase N, Annexin 5A, Annexin A2, AP-1, ATP citrate lyase, ATPase, Basigin, Caveolin-1, Clathrin, Claudin-1, Cofilin-1, EGFR, Ep-CAM, ICAM, HLA-ABC, prostate specific antigen, Rab-14, Rab-7, Syndecan, Tumor-Associated Glycoprotein, Tetraspanin-8, Tsg101, vacuolar-sorting protein 35, CD2, CD3, CD5, CD8, CD9, CD11a, CD11b, CD11c, CD13, CD29, CD37, CD41, CD44, CD49d, CD49f, CD62L, CD63, CD68, CD80, CD81, CD86, CD90, CD142, CD146, CD163, CD192, and CD202b.

Specific examples of markers that may associate a disease such as a viral infection with a specific organ or tissue include surfactant associated protein A (SP-A) and surfactant associated protein B (SP-B) for the lung, cardiac troponin for the heart, von Willebrand factor and CD31/PECAM-1 for endothelium, Enolase-2 (ENO2) and neuron specific enolase (NSE) for the brain or neuro tissues, Asialoglycoproteinreceptor 1 (ASGR-1) for the liver, and Aquaporin 6 for the kidney. These markers are particularly suitable for associating a virus infection with a specific organ or tissue as origin of virus replication, more preferably for associating a SARS-CoV virus or Influenza A/B virus infection with a specific organ or tissue, and most preferably a SARS-CoV 2 virus infection.

Analyzing the content of the exosome may include lysis of the exosomes. The content to be analyzed include but are not limited to peptides, proteins, microRNA, DNA, and/or RNA such as mRNA. Proteins to be analyzed typically include but are not limited to platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. The analysis of the contents may include DNA mutation analysis, RNA expression, DNA methylation quantification and/or protein expression, as well as fluorescence flow cytometry. In case of analyzing nucleic acids, these can be for example quantified to identify their profiles by methods known in the field and involving for example RT-PCR. Exosomes which are released from virus infected cells contain Rab11 which allows virosome identification and applying a specific second antibody directed against a specific viral protein for diagnosing and monitoring viral diseases. The analysis of the exosome contents and surface proteins and peptides is not limited to detecting and monitoring cancer or viral infections but may be generally used for detecting or characterizing many medical conditions and diseases.

According to a further aspect, the present invention provides a kit for performing any of the methods of the present invention as described herein. The kit comprises a first binding agent that specifically binds to the extra-vesicular part of Rab11 or a second binding agent that specifically binds to the extra-vesicular part of Rab4, or a combination thereof; and instructions for using the first and/or the second binding agent for binding of said first and/or second binding agent to exosomes in a sample. The binding agents are as defined above and may comprise a label. Alternatively, the kit may further comprise a third binding agent as defined herein above, which third binding agent comprises a label as defined above. The kit may further comprise means and/or instructions for preparing the sample before the binding agent(s) is/are added to the sample.

According to a further aspect, the present invention provides a method of diagnosing cancer or a viral infection in a subject. Analogous to the methods described above, the method of diagnosing cancer or a viral infection in a subject comprises the steps of a) obtaining a sample from a person known to or suspected of having cancer or a viral infection, b) producing an exosome enriched fraction from the sample, and c) detecting tumor-related or viral-related exosomes in the exosome enriched fraction of step a) with at least one binding agent specifically binding to a tumor antigen or viral antigen. Step b) further comprises the steps of i) contacting a first binding agent that specifically binds to the extra-vesicular part of Rab11 or a second binding agent that specifically binds to the extra-vesicular part of Rab4, or a combination thereof, with the sample under conditions allowing binding of said first binding agent and/or second binding agent to exosomes, and ii) separating exosomes to which the first binding agent and/or the second binding agent is bound from the sample. The sample, the binding agents, the tumor antigens or viral antigens, and the conditions allowing binding of said binding agents are as defined above. Accordingly, the sample can be a body fluid. Preferably, the body fluid is selected from the group consisting of plasma, ascites, cerebral fluid, bone marrow, urine, faeces or bronco-alveolar washing. The binding agent can be an antigen binding agent, preferably, the antigen binding agent is an antibody. The tumor antigen is preferably selected from the group consisting of but not limited to GPER-1, CD247, and phosphatidylserine.

The method may, according to an embodiment, further comprise—prior to the step of producing an exosome enriched fraction from the sample—the optional step of suspending or solubilizing the sample. The method may further comprise enriching exosomes from the sample based on size and/or density. However, such enriching is not necessary and the method of the invention can be performed directly on the sample as is shown for example in Example 11 below.

According to a further embodiment, step ii) comprises the step of performing flow cytometry or chromatography as defined above.

According to a preferred embodiment, the first binding agent comprises a first label and/or the second binding agent comprises a second label. Alternatively, the first binding agent and/or the second binding agent is bound by a third binding agent specifically binding to the first binding agent and/or the second binding agent, wherein the third binding agent comprises a third label. The labels in this embodiment are as defined above. Accordingly, the first, second and third label is independently selected from the group consisting of an enzyme label, a fluorescence label, a radioactive label, a magnetic label, a peptide or protein label, or a quantum dot.

According to an alternative embodiment, the first binding agent and/or second binding agent is covalently or non-covalently bound on a solid surface.

According to a preferred embodiment, the first binding agent binds to the extra-vesicular part of Rab11A and the second binding agent binds to the extra-vesicular part of Rab4A.

The invention is described by way of the following examples which are to be construed as merely illustrative and not limitative of the scope of the invention.

### EXAMPLES

### Devices and Antibodies

In the examples of the invention, the following devices and antibodies have been used: Centrifuge: Optima LE-80K von Beckman Coulter
FACS device: Sony SP6800 Spectral Analyser, Sony SA Spectral Analyser
Pancoll gradient: PAN Biotech, Density: 1.077 g/ml
PEG6000: Molecular Biology grade, Merck Antibodies with indicated labels: against HLA-ABC-PE-Cy5, CD5-FITC, CD8-PE-Cy7, CD13-BV421, CD81-PE-Dazzle 594, CD41-PacBlue CD68-FITC, CD86-BV650, GPER-1-DyeLight 405, Rab5-PE.
The antibody used in the examples against Rab11 is a rabbit polyclonal antibody labeled with phycoerythrin (PE) obtained from Biorbyt, Ltd., UK, order number orb484348 (Rab11-PE).
The antibody used in the examples against Rab4 is a rabbit polyclonal antibody labeled with phycoerythrin (PE) obtained from Biorbyt, Ltd., UK, order number orb496526 (Rab4-PE).

### Example 1

1. Plasma Isolation from Whole Blood Using Pancoll Gradient

Whole blood was drawn from donors into EDTA-containing blood collection tubes. The blood was transferred into 50 ml tubes and 20 ml of whole blood was mixed with PBS to a volume of 37.5 ml before 12.5 ml Pancoll was layered at the bottom of the tubes. The samples were centrifuged at 1,000 g for 17 min with deceleration speed set to 2 and acceleration speed set to 7. The top layer, resembling the blood plasma, was transferred into a new tube, centrifuged at 2,000 g for 15 min and the supernatant was transferred into 2 ml collection tubes. The samples were centrifuged at 10,000 g and 4° C. for 10 min, the supernatants were transferred into a 50 ml tube and stored on ice until analysis. →Plasma (10,000 g) sample.

### 2. Exosome Enrichment

### a) Exosome Enrichment Via Ultracentrifugation

Plasma isolated via Pancoll gradient was used for the enrichment of exosomes by ultracentrifugation. 4 ml plasma was transferred into ultracentrifugation tubes and spun at 100,000 g for 1.5 h at 4° C. The supernatant was discarded and the pellet (not visible) was resuspended by adding 400 μl 1×PBS and incubation at 4° C. for at least half an hour during which the tubes were vortexed briefly or the liquid was pipetted up and down. Following, the resuspended exosomes were stored on ice until analysis. →UC (100,000 g, 4° C.) sample.

### b) Exosome Enrichment Using the Total Exosome Isolation Kit (Invitrogen)

Plasma isolated via Pancoll gradient was used for the enrichment of exosomes. 2 ml of plasma was transferred into a reaction tube and mixed with 400 μl Total exosome isolation reagent. After incubation for 10 min at room temperature the sample was centrifuged for 5 min at 10.000 g and room temperature. Finally, the exosome-containing pellet was resuspended in 200 μl PBS and stored on ice until analysis→Total Exosome Isolation sample

### c) Exosome Enrichment Using the ExoSpin Kit (CellGuidance Systems)

Plasma isolated via Pancoll gradient was used for the enrichment of exosomes. 1 ml of plasma was transferred into a reaction tube and mixed with 500 μl buffer. After incubation for 60 min at 4° C. the sample was centrifuged for 60 min at 16.000 g and room temperature. The exosome-containing pellet was resuspended in 200 μl PBS and 100 μl were loaded on 2 columns each. Exosomes were eluted from the columns with 100 μl reagent and stored on ice until analysis→ExoSpin sample.

### d) Exosome Enrichment Using PEG6000

Plasma isolated via Pancoll gradient was used for the enrichment of exosomes. 4 ml of plasma was transferred into a reaction tube and mixed with 4 ml PEG6000 (16%, 1 mM NaCl; final concentration of 8% PEG6000 and 0.5 mM NaCl). After incubation for 12 at 4° C. the sample was centrifuged for 60 min at 3.000 g and 4° C. The exosome-containing pellet was resuspended in 400 μl PBS and stored on ice until analysis→PEG6000 sample.

e) Exosome Enrichment Using the exoEasy Maxi Kit (Qiagen)

For the enrichment of exosomes from plasma isolated via Pancoll gradient, the exoEasy Maxi Kit from Qiagen was used according to the instruction manual using 4 ml human plasma. The exosomes were eluted in 400 μl elution buffer and stored on ice until analysis→ExoEasy sample.

### 3. Exosome Staining and FACS Analysis

For staining the enriched exosomes, 2×50 μl of each of the above test procedures a) to e) were transferred into a 96-well plate and fluorescently labelled antibodies (Rab5-PE, CD41-PacificBlue, CD81-PE/Dazzle594, HLA-ABC-PE/Cy5) were added to one well each. The samples were incubated for 30 min on ice in the dark before they were analyzed by flow-cytometry. The threshold value was set to 0.1% to make sure not to lose any exosomes and the sample flow rate was set not higher than 2 to guarantee that only single exosomes pass the laser beam.

### 4. Results

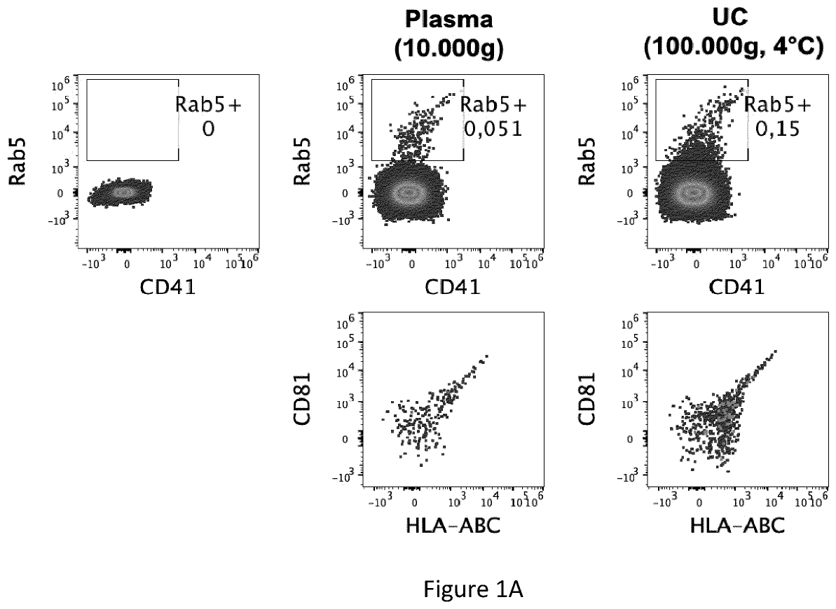

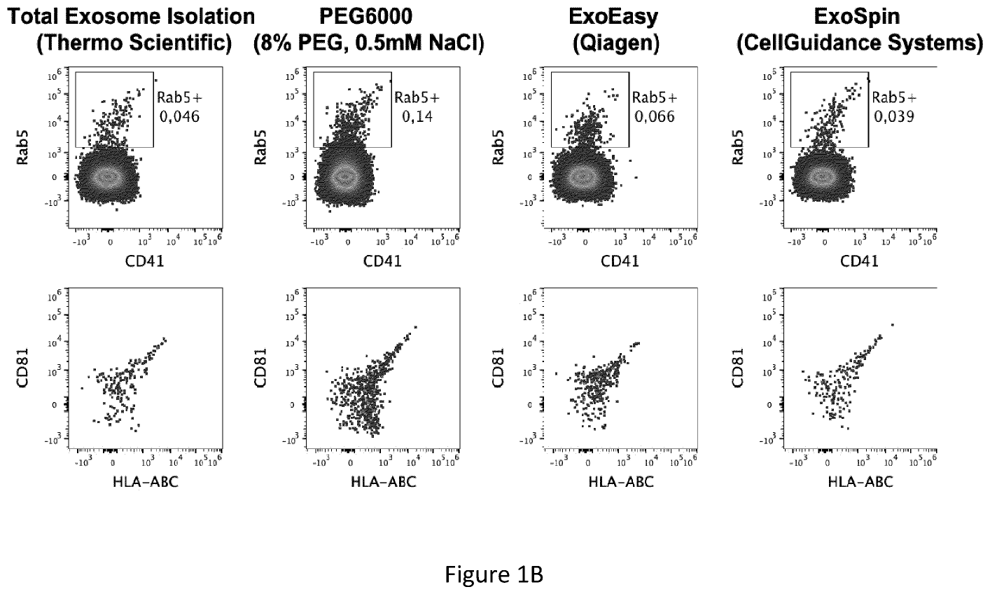

FIGS. 1A and 1B summarize the results of the experiments. Gates in the graphs depict the exosome fractions. The number of exosomes is not significantly increased in any of the tested methods compared to the number of exosomes enriched from untreated plasma. Hence, the currently available methods are insufficient for enriching, isolating or purifying exosomes.

### Example 2

1. Plasma Isolation from Whole Blood Using Pancoll Gradient

Whole blood was drawn from donors into EDTA-containing blood collection tubes. The blood was transferred into 50 ml tubes and 20 ml of whole blood was mixed with PBS to a volume of 37.5 ml before 12.5 ml Pancoll was layered at the bottom of the tubes. The samples were centrifuged at 1,000 g for 17 min with deceleration speed set to 2 and acceleration speed set to 7. The top layer, resembling the blood plasma, was transferred into a new tube, centrifuged at 2,000 g for 15 min and the supernatant was transferred into 2 ml collection tubes. The samples were centrifuged at 10,000 g and 4° C. for 10 min, the supernatants were transferred into a 50 ml tube and stored on ice until analysis→Plasma (10,000 g) sample.

### 2. Exosome Enrichment

### a) Exosome Enrichment Via Ultracentrifugation

Plasma isolated via Pancoll gradient was used for the enrichment of exosomes by ultracentrifugation. 4 ml plasma was transferred into ultracentrifugation tubes and spun at 100,000 g for 1.5 h at 4° C. The supernatant was discarded and the pellet (not visible) was resuspended by adding 400 μl 1×PBS and incubation at 4° C. for at least half an hour during which the tubes were vortexed briefly or the liquid was pipetted up and down. Subsequently, the resuspended exosomes were stored on ice until further analysis→UC (100,000 g, 4° C.) sample.

### b) Exosome Enrichment Using PEG6000

Plasma isolated via Pancoll gradient was used for the enrichment of exosomes. 4 ml of plasma was transferred into a reaction tube and mixed with 4 ml PEG6000 (16%, 1 mM NaCl; final concentration of 8% PEG6000 and 0.5 mM NaCl). After incubation for 12 at 4° C. the sample was centrifuged for 60 min at 3,000 g and 4° C. The exosome-containing pellet was resuspended in 400 μl PBS and stored on ice until further analysis→PEG6000 sample.

### 3. Exosome Staining and FACS Analysis

For staining exosomes in the samples from plasma, 2×50 μl each were transferred into a 96-well plate and fluorescently labelled antibodies (Rab5-PE, CD41-PacificBlue, CD81-PE/Dazzle594, HLA-ABC-PE/Cy5) were added to one well each. The samples were incubated for 30 min on ice in the dark before they were analyzed by flow-cytometry. The threshold value was set to 0.1% to make sure not to lose any exosomes and the sample flow rate was set not higher than 2 to guarantee that only single exosomes pass the laser beam.

### 4. Results

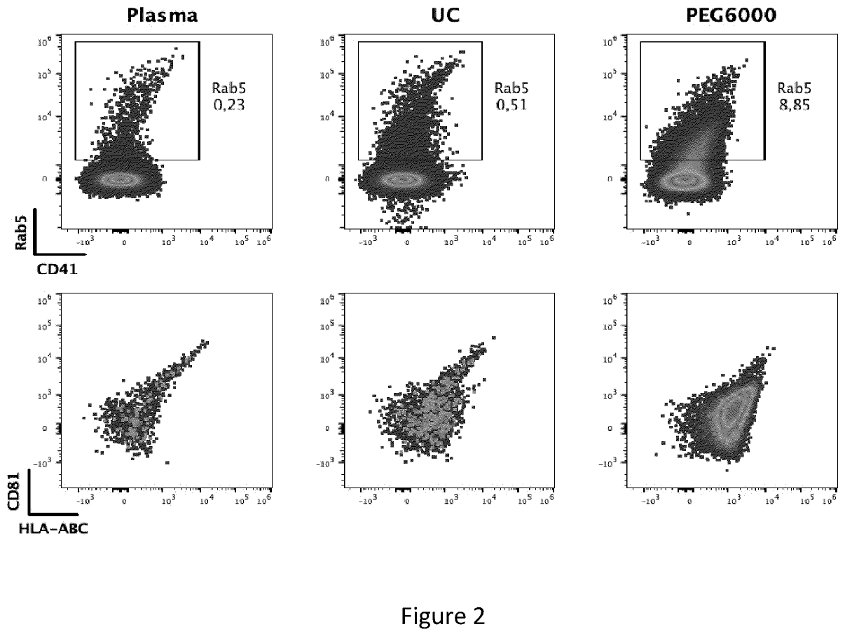

FIG. 2 summarizes the results of the experiments. Gates in the graphs depict the exosome fractions. From left to right: plasma sample; ultracentrifugation sample, PEG6000 sample. Precipitation with PEG6000 and subsequent Rab5 staining gives the highest exosome quantity of all conventional methods tested.

### Example 3

### 1. Exosome Enrichment

a) Exosome Enrichment from Plasma Via Ultracentrifugation

Plasma isolated via Pancoll gradient (cf. examples 1 and 2) was used for the enrichment of exosomes by ultracentrifugation. 10 ml plasma was transferred into ultracentrifugation tubes and spun at 100,000 g for 1.5 h at 4° C. The supernatant was discarded and the pellet (not visible) was resuspended by adding 500 μl 1×PBS and incubation at 4° C. for at least half an hour during which the tubes were vortexed briefly or the liquid was pipetted up and down. Subsequently, the resuspended exosomes were stored on ice until analysis→Plasma sample.

b) Exosome Enrichment from HL-60 Supernatant (Cancer Cell Line Supernatant) Via Ultracentrifugation

HL-60 (human leukemia) cell culture supernatant from a 3 day culture was used for the enrichment of exosomes by ultracentrifugation. Supernatant was centrifuged at 2,000 g for 15 min and transferred into 2 ml collection tubes. The samples were centrifuged at 10,000 g and 4° C. for 10 min, 10 ml were transferred into ultracentrifugation tubes and spun at 100,000 g for 1.5 h at 4° C. The supernatant was discarded and the pellet (not visible) was resuspended by adding 500 μl 1×PBS and incubation at 4° C. for at least half an hour during which the tubes were vortexed briefly or the liquid was pipetted up and down. Following, the resuspended exosomes were stored on ice until analysis→Cancer cell line supernatant sample.

c) Exosome Enrichment from HER2 (CD340) Breast Cancer Cell Line

BT474 cell culture (human invasive ductal carcinoma of the breast) supernatant from day 3 culture was used for the analysis of the exosomes. Supernatant was centrifuged at 10000 g for 10 min. 200 μl were stained using anti-Rab11-PE antibody and anti-CD340-FITC antibody. The samples were incubated for 30 min on ice in the dark before they were analyzed by flow cytometry. Trashold value was set to 0.1% (SSC).

### 2. Exosome Staining and FACS Analysis

For staining exosomes in the samples from plasma and the cancer cell line, 2×50 μl each were transferred into a 96-well plate and fluorescently labelled antibodies (Rab5-PE, Rab4-PE or Rab11-PE) were added to one well each. The samples were incubated for 30 min on ice in the dark before they were analyzed by flow-cytometry. The threshold value was set to 0.1% to make sure not to lose any exosomes and the sample flow rate was set not higher than 2 to guarantee that only single exosomes pass the laser beam.

### 3. Results

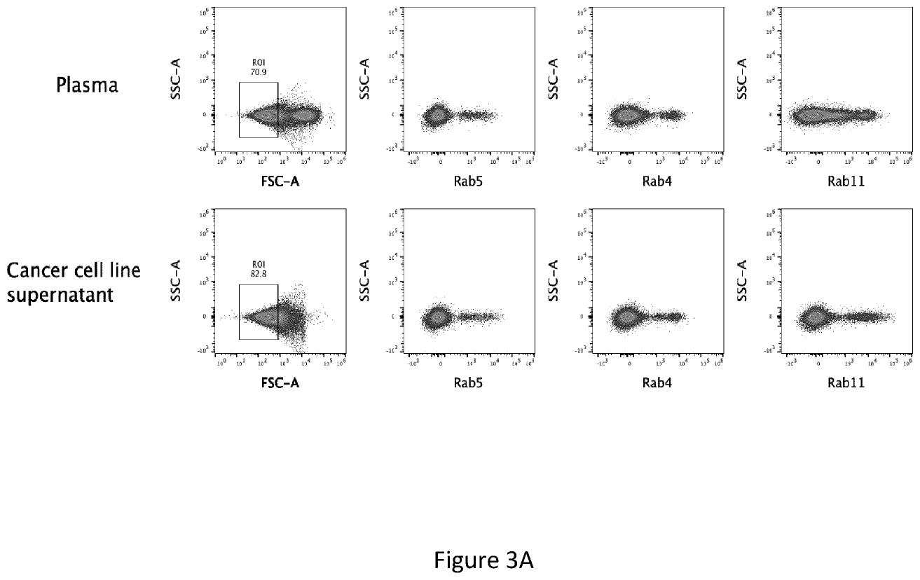

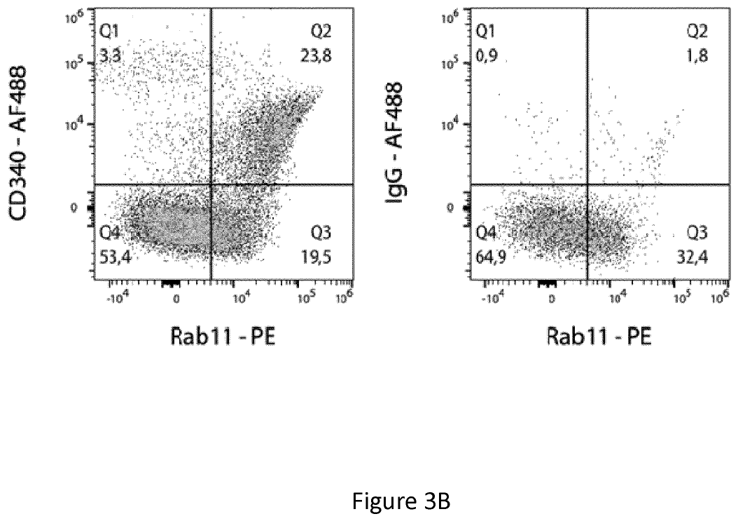

FIG. 3A shows identification of Rab4+, Rab11+ and Rab5+ exosomes, respectively, from plasma and cancer cell line supernatants. Using Rab4 or Rab11 staining results in higher exosome quantity than using Rab5 staining (region of interest, ROI). FIG. 3B shows detection of CD340 (HER2) on Rab11 positive exosomes by means of flow cytometry (left panel, right upper quadrant). Control antibody with Rab11 positive exosomes does not show any detection (right panel, right upper quadrant empty).

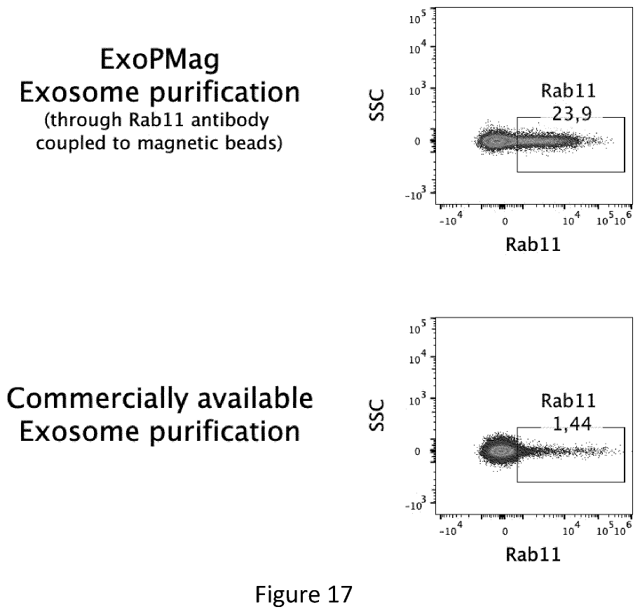

FIG. 17 additionally shows that Rab11 is superior in purifying exosomes from human plasma compared to an isolation using Qiagen ExoEasy Purification kit, which is based on water deprivation and precipitation. Exosomes were purified (upper panel) with Rab11 antibody coupled to magnetic beads from human plasma and analyzed by fluorescence flow cytometry. Lower panel shows exosomes purified from the same plasma sample with Qiagen's ExoEasy Isolation kit and subsequent analysis by fluorescence flow cytometry. Rab11 surprisingly mediated purification yields approximately 16-fold higher amounts of exosomes. In addition, using flow cytometry and sorting with Rab11 (here by applying an anti-Rab11 antibody in plasma from healthy volunteers), exosomes can be physically separated from other microvesicles (table 1). The results show that Rab 11 antibody detects CD41 negative EVs but not CD41 positive microvesicles. Isolating EVs based on Rab11 thus leads to isolation of pure exosomes without contamination with platelet-derived microvesicles.

TABLE 1
Characterization of plasma, sorted for CD41positive microvesicles
(platelet marker) vs. Rab11positive exosomes

| Plasma | Staining | Staining | Staining |
|---|---|---|---|
| sorted for | Rab11 | CD41 | CD9 |
| CD41^{positive} vesicles | 0.0% | — | 28.9% |
| Rab11^{positive} vesicles | — | 0.0% | 4.0% |

### Example 4

### 1. Exosome Enrichment

Exosome enrichment was carried out as identified above from plasma using ultra-centrifugation and PEG6000 (example 2).

### 2. Exosome Staining and FACS Analysis

Exosome staining and FACS analysis was carried out as identified above (example 3) with fluorescently labelled antibodies against Rab4-PE.

### 3. Results

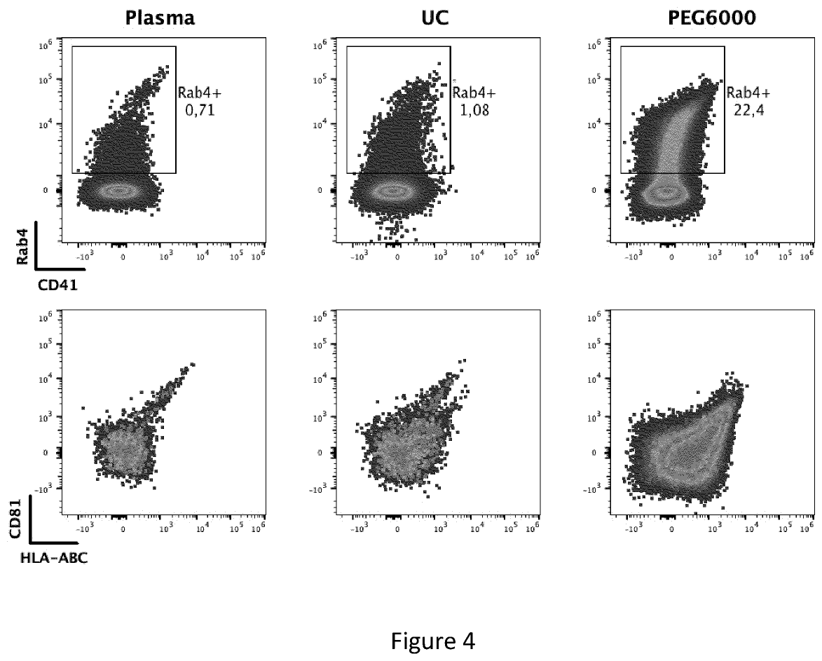

FIG. 4 shows identification of Rab4+ exosomes in the plasma sample, in the ultra-centrifugation sample and in the PEG6000 sample. Rab4 staining results in higher exosome quantity than using Rab5 staining in comparable samples (example 2).

### Example 5

### 1. Exosome Enrichment

Exosome enrichment was carried out as identified above from plasma using ultra-centrifugation and PEG6000 (example 2).

### 2. Exosome Staining and FACS Analysis

Exosome staining and FACS analysis was carried out as identified above (example 3) with fluorescently labelled antibodies against Rab11-PE.

### 3. Results

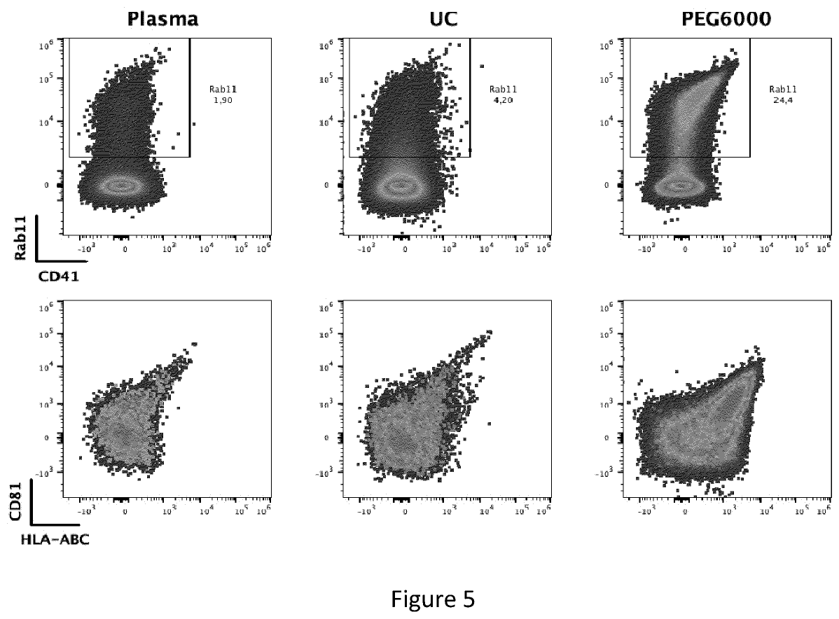

FIG. 5 shows identification of Rab11+ exosomes in the plasma sample, in the ultra-centrifugation sample and in the PEG6000 sample. Rab11 staining results in higher exosome quantity than using Rab5 or Rab4 staining in comparable samples (examples 2 and 4).

### Example 6

### 1. Exosome Enrichment

Exosome enrichment was carried out as identified above in example 1 a) to e).

### 2. Exosome Staining and FACS Analysis

Exosome staining and FACS analysis was carried out as identified in example 1 with fluorescently labelled antibodies against Rab11-PE.

### 3. Results

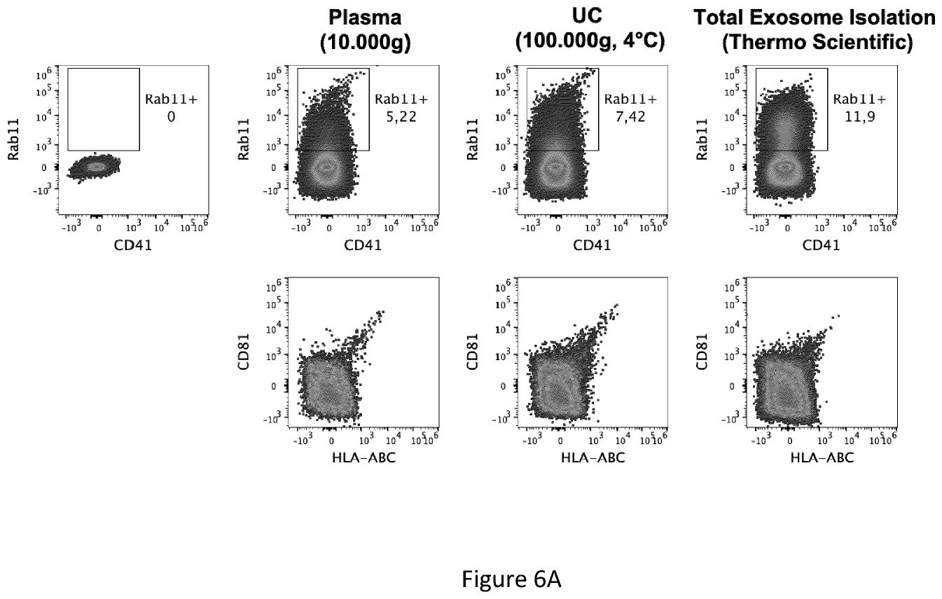

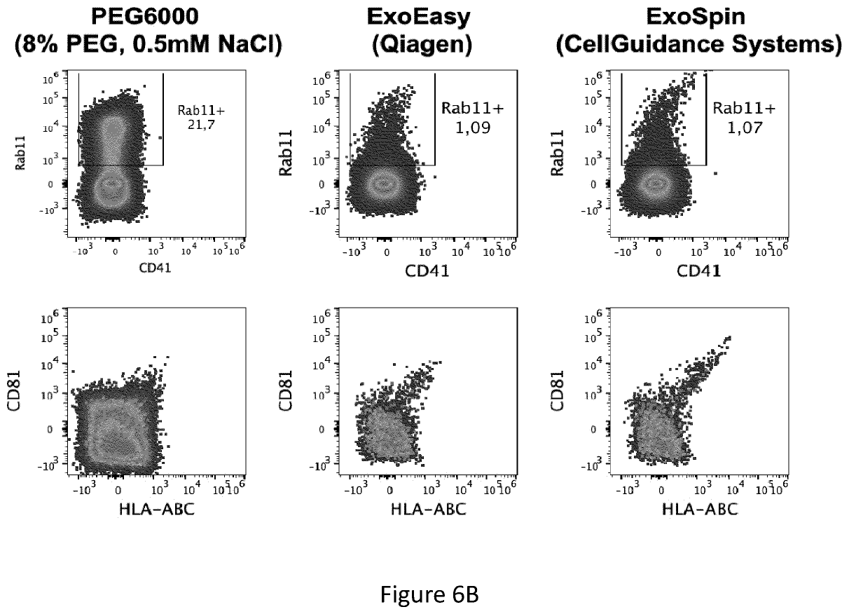

FIGS. 6A and 6B show identification of Rab11+ exosomes in the plasma sample and in samples prepared by the conventional methods. Using Rab11 staining significantly improves exosome quantity in all samples tested (compare with example 1; FIGS. 1A and 1B). Unstained sample was used as internal control.

### Example 7

### 1. Exosome Enrichment

Exosome enrichment was carried out as identified above in example 1 a) to e). Exosomes were enriched by ultracentrifugation of Plasma.

### 2. Exosome Staining and FACS Analysis

Exosome staining and FACS analysis was carried out as identified in example 1 a) to e). with fluorescently labelled antibodies against Rab11-FITC (Biorbyt, Ltd., UK), Rab4-PE and Rab5-PE.

### 3. Results

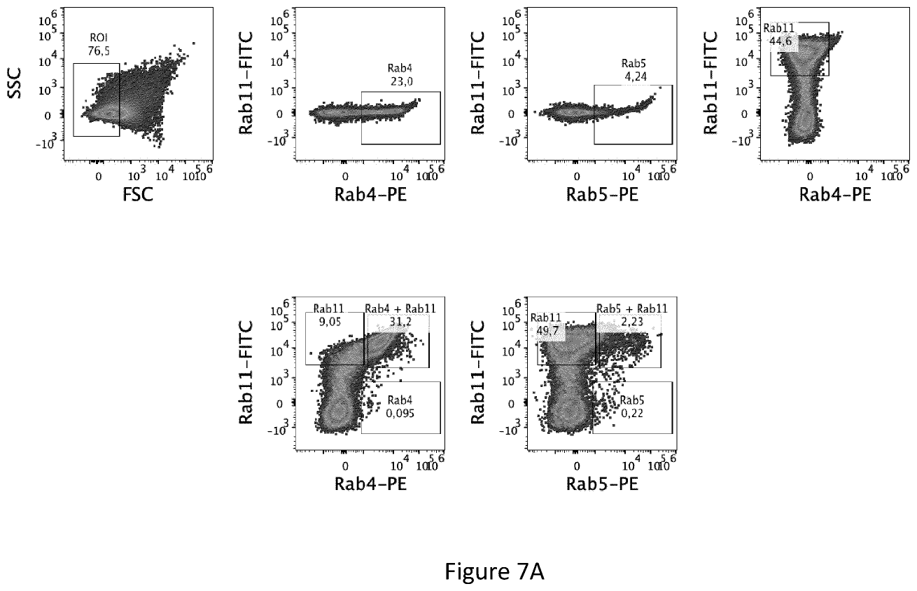

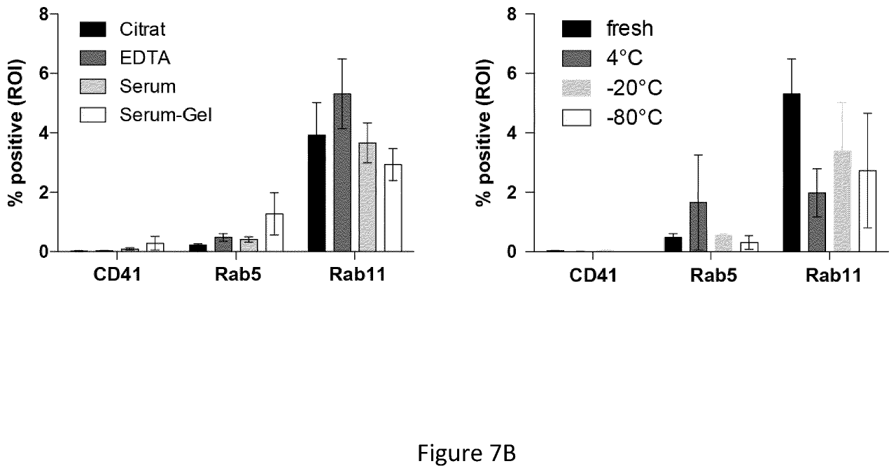

FIG. 7A shows that Rab4 and Rab5 staining only identify exosome subpopulations. In contrast, Rab11 staining enables identification of more exosomes, including Rab4 and Rab5 positive exosome populations. FIG. 7B shows that Rab11 demonstrates the highest yields under all conditions tested (left figure). Surprisingly Rab11 outperforms Rab5 mediated purification even under all storage conditions tested (right figure). Serum and plasma obtained from one healthy volunteer with different anticoagulants results in different yields (left). Yield is also dependent on storage conditions (right). CD41 serves as control to confirm that exosomes are not derived from platelets.

### Example 8

### 1. Exosome Enrichment

a) Exosome Enrichment from HL-60 and SKBR-3 Supernatant Via Ultracentrifugation

HL-60 (human leukemia) and SKBR-3 (breast cancer) cell culture supernatant from a 2 day culture was used for the enrichment of exosomes by ultracentrifugation. Supernatant from each cell line was centrifuged at 2,000 g for 15 min and transferred into 2 ml collection tubes. The samples were centrifuged at 10,000 g and 4° C. for 10 min, 40 ml supernatant from each cell line were transferred into ultracentrifugation tubes and spun at 100,000 g for 1.5 h at 4° C. The supernatant was discarded and the pellet (not visible) was resuspended by adding 4 ml 1×PBS per cell line and incubation at 4° C. for at least half an hour during which the tubes were vortexed briefly or the liquid was pipetted up and down. Subsequently, the resuspended exosomes were stored on ice until further analysis.

### 2. Exosome Staining and FACS Analysis

For staining the isolated exosomes from cell culture supernatant, 2×50 μl each were transferred into a 96-well plate and fluorescently labelled antibodies (listed in table 2) were added to one well each. The samples were incubated for 30 min on ice in the dark before they were analyzed by flow-cytometry. The threshold value was set to 0.1% to make sure not to lose any exosomes and the sample flow rate was set not higher than 2 to guarantee that only single exosomes pass the laser beam.

TABLE 2
Antibody stainings used for exosomal surface mapping

| Stainings | Antibodies |
|---|---|
| #1 | Rab4-, Rab5- or Rab11-PE, CD9-FITC, |
| | CD41-PacificBlue, CD81-PE/Dazzle594, |
| | CD63-BV421, HLA-ABC-PE/Cy5 |
| #2 | Rab4-, Rab5- or Rab11-PE, CD1a-eF450, |
| | CD1c-FITC, CD2-PE/Cy5, CD3-PE/Dazzle594, |
| | CD4-PE/Cy7 |
| #3 | Rab4-, Rab5- or Rab11-PE, CD5-FITC, |
| | CD8-PE/Cy7, CD13-BV421 |
| #4 | Rab4-, Rab5- or Rab11-PE, CD11b-PE/Dazzle594, |
| | CD11c-PE/Cy7, CD7-FITC, CD15-PerCpCy5.5, |
| | CD16-eF450 |
| #5 | Rab4-, Rab5- or Rab11-PE, CD28-AF488, |
| | CD33-eF450, CD34-PE/Cy7 |
| #6 | Rab4-, Rab5- or Rab11-PE, CD24-BV510, |
| | CD25-PerCpCy5.5, CD38-eF450, CD40-BV421, |
| | CD43-PE/Cy7 |
| #7 | Rab4-, Rab5- or Rab11-PE, CD45-PE/Cy7, |
| | CD45RA-PerCpCy5.5, CD62L-eF450, |
| | CD45RO-PE/Dazzle594 |
| #8 | Rab4-, Rab5- or Rab11-PE, CD49d-FITC, |
| | CD56-BV711,CD69-BV605, CD73-BV421, |
| | CD83-PE/Cy7 |
| #9 | Rab4-, Rab5- or Rab11-PE, CD68-FITC, |
| | CD80-BV421, CD86-BV650, CD95-PE/Dazzle594, |
| | CD96-PE/Cy7 |
| #10 | Rab4-, Rab5- or Rab11-PE, CD90-FITC, |
| | CD107a-BV650, CD123-PE/Cy7, CD127-eF450 |
| #11 | Rab4-, Rab5- or Rab11-PE, CD94-FITC, |
| | CD117-BV650, CD158-PerCpCy5.5 |
| #12 | Rab4-, Rab5- or Rab11-PE, CD134-FITC, |
| | CD154-BV605, CD186-PE/Cy7 |
| #13 | Rab4-, Rab5- or Rab11-PE, CD146-FITC, |
| | CD183-BV711, CD192-BV421, CD194-BV605, |
| | CD196-PE/Cy7 |
| #14 | Rab4-, Rab5- or Rab11-PE, CD197-BV711, |
| | CD202b-AF488, CD274-BV421, CD336-PerCpCy5.5 |
| #15 | Rab4-, Rab5- or Rab11-PE, CD226-FITC, |
| | CD304-BV421, CD357-PE/Cy5 |
| #16 | Rab4-, Rab5- or Rab11-PE, CD247-FITC, |
| | CXCR1-PerCpCy5.5 |
| #17 | Rab4-, Rab5- or Rab11-PE, CD335-FITC, |
| | CD360-BV421, Dectin1-PerCpeF710 |
| #18 | Rab4-, Rab5- or Rab11-PE, LAG3-AF488, |
| | LAP-PE/Cy7 |

### 3. Results

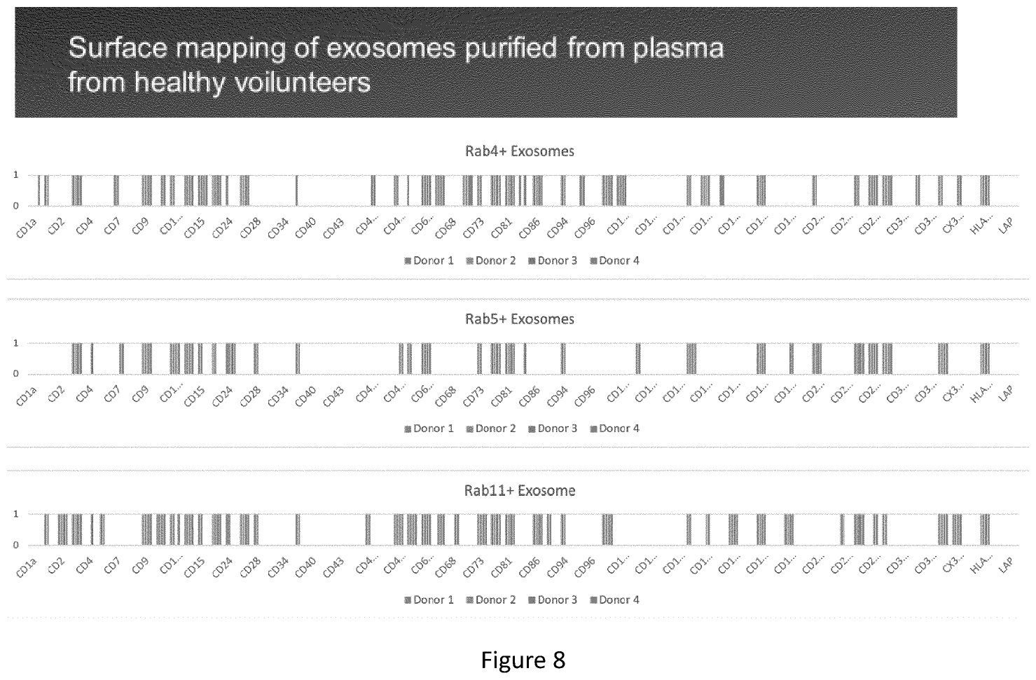

FIG. 8 shows surface mapping of exosomes purified from plasma samples of healthy volunteers. For the mapping, exosomes were stained with either Rab4-PE, Rab5-PE or Rab11-PE antibodies together with different fluorescently labelled antibodies (see table 2). Rab4-, Rab5- or Rab11-positive exosomes were analyzed for additional signals of the other tested markers. Markers that showed positive staining on Rab4-, Rab5- or Rab11-positive exosomes were rated as “1”, markers that were negative were rated as “0”. These results were combined and transferred into one map.

### Example 9

1. Plasma Isolation from Whole Blood Using Pancoll Gradient

Whole blood was drawn from donors into EDTA-containing blood collection tubes. The blood was transferred into 50 ml tubes and 20 ml of whole blood was mixed with PBS to a volume of 37.5 ml before 12.5 ml Pancoll was layered at the bottom of the tubes. The samples were centrifuged at 1,000 g for 17 min with deceleration speed set to 2 and acceleration speed set to 7. The top layer, resembling the blood plasma, was transferred into a new tube, centrifuged at 2,000 g for 15 min and the supernatant was transferred into 2 ml collection tubes. The samples were centrifuged at 10,000 g and 4° C. for 10 min, the supernatants were transferred into a 50 ml tube and stored on ice until further analysis.

### 2. Exosome Enrichment Via Ultracentrifugation

Plasma isolated via Pancoll gradient was used for the enrichment of exosomes by ultracentrifugation. 40 ml plasma was transferred into ultracentrifugation tubes and spun at 100,000 g for 1.5 h at 4° C. The supernatant was discarded and the pellet (not visible) was resuspended by adding 4 ml 1×PBS and incubation at 4° C. for at least half an hour during which the tubes were vortexed briefly or the liquid was pipetted up and down. Subsequently, the resuspended exosomes were stored on ice until further analysis.

### 3. Exosome Staining and FACS Analysis

For staining the isolated exosomes from plasma, 2×50 μl each were transferred into a 96-well plate and fluorescently labelled antibodies were added to one well each. The samples were incubated for 30 min on ice in the dark before they were analyzed by flow-cytometry. The threshold value was set to 0.1% to make sure not to lose any exosomes and the sample flow rate was set not higher than 2 to guarantee that only single exosomes pass the laser beam.

### 4. Results

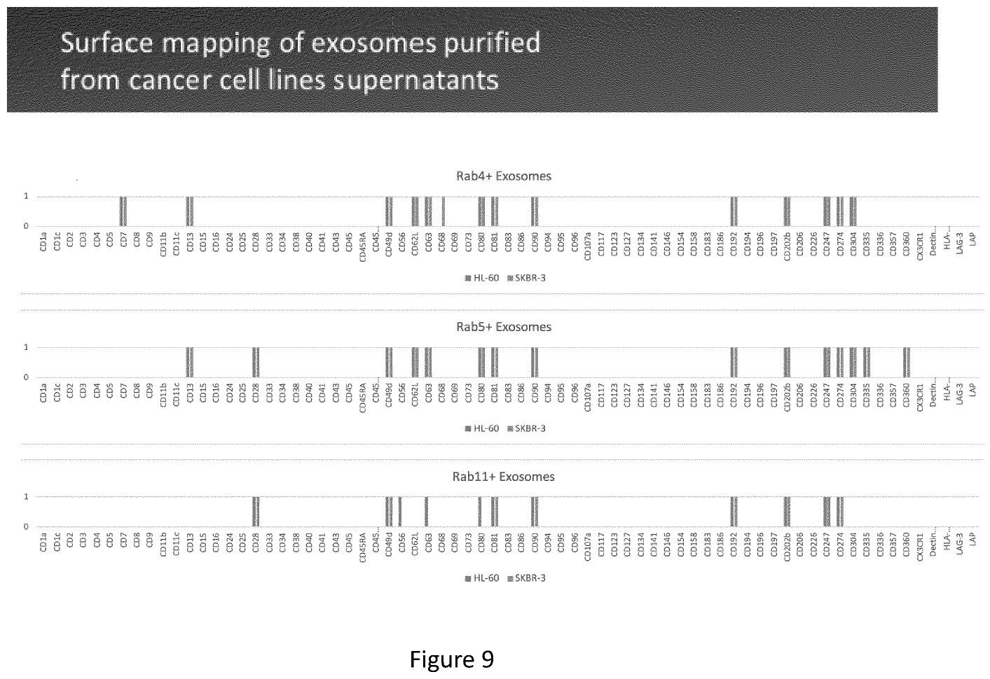

FIG. 9 shows surface mapping of exosomes purified from cancer cell line samples.

The following table 3 summarizes the results obtained in examples 8 and 9.

TABLE 3
Exosome mapping - data of 7 donors

| | | Surface | |
|---|---|---|---|
| Surface markers | Surface | markers | |
| exosomes | markers | unique to | |
| from Healthy | exosomes from | exosomes from | Surface marker |
| Volunteers | Cancer Cells | Cancer Cells | family |
| CD3 (7) | CD49d | CD49d* | Integrin α2β1 |
| CD9 (4) | CD81 | CD90* | Thy-1 membrane |
| | | | glycoprotein |
| CD13 (5) | CD90 | CD202b* | Angiopoietin-1 |
| | | | receptor |
| CD62L (6) | CD192 | CD247 | T-cell receptor |
| | | | T3 zeta chain |
| CD80 (6) | CD202b | CD274* | PD-L1 |
| CD81 (7) | CD247 | | |
| CD192 (5) | CD274 | | |
| HLA-ABC (7) | | | |
| 8 (3) | 7 | 5 (1) | |
| bold = marker | | bold = compared | |
| detectable | | with all 7 donors | |
| with 7 donors | | | |

Bold font: overlap considering data from 7 donors; *detected at least for 1 donor of the first measurement
Final check of validity of uniqueness can be performed with percentage of signals per marker and donor
Cancer cells: HL60 (human leukemia cell line) and SKBR3 (breast cancer cell line)

### Example 10

T-distributed Stochastic Neighbor Embedding (t-SNE) analysis of exosomes from healthy volunteers was performed. t-SNE is a machine learning algorithm for visualization. Using heatmap color-code for each marker, the expression of various markers in different populations is possible. In the present example it is used to visualize high-level representations learned by an artificial neural network. The analysis revealed that by using antibodies against Rab11, more exosomes can be identified than using antibodies against Rab4 or Rab5, which finding further confirms the results of examples 3 to 7.

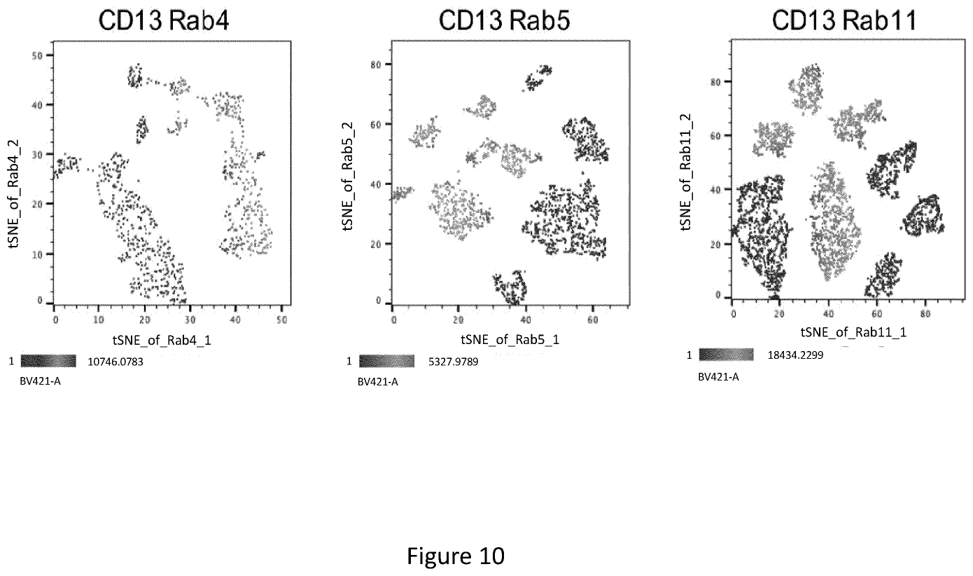

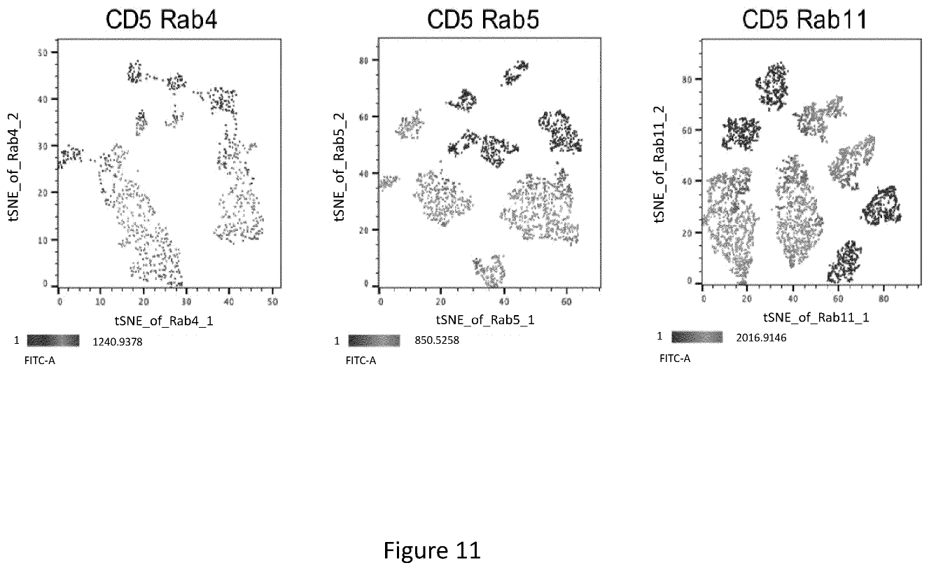

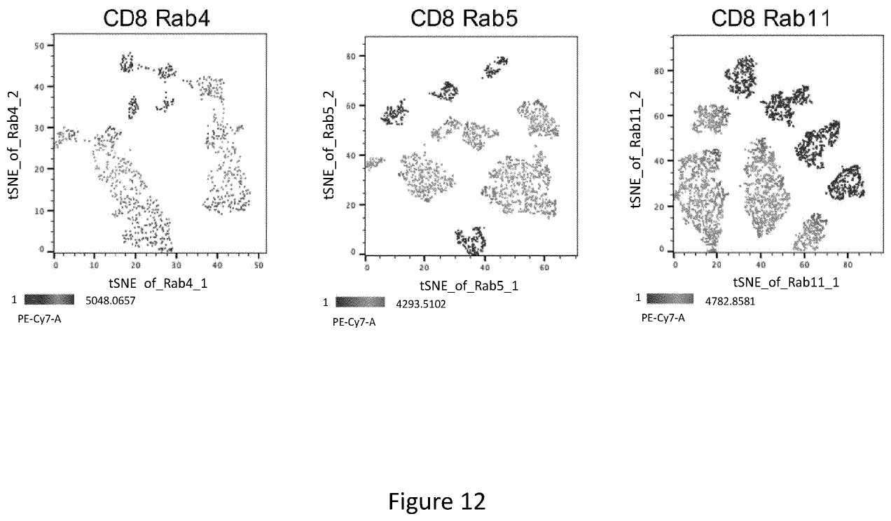

FIG. 10 shows staining in CD13 exosomes with Rab4, Rab5 and Rab11; FIG. 11 shows staining in CD5 exosomes with Rab4, Rab5 and Rab11; and FIG. 12 shows staining in CD8 exosomes with Rab4, Rab5 and Rab11.

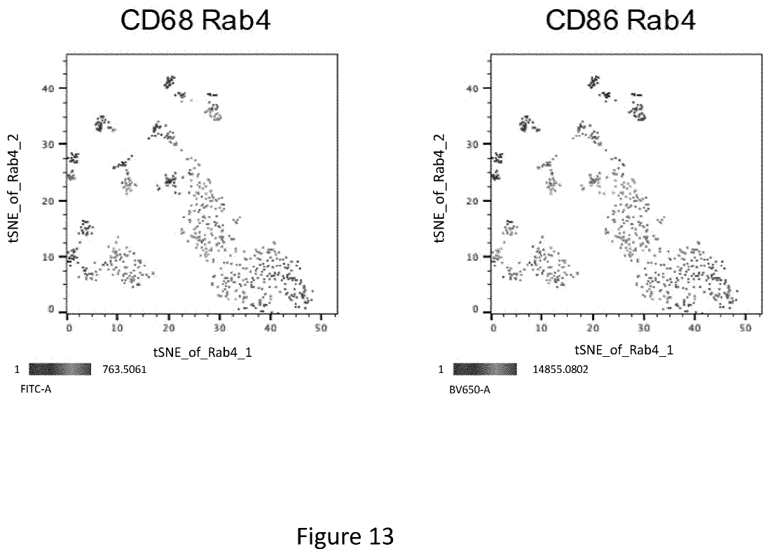

FIG. 13 shows staining in CD68 and CD86 exosomes with Rab4. Rab4 identifies CD68 positive exosomes which are also CD86 positive.

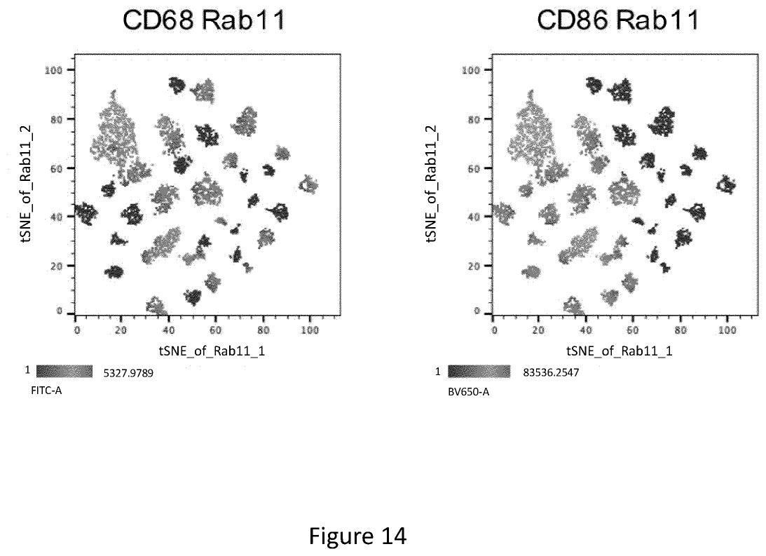

FIG. 14 shows staining in CD68 and CD86 exosomes with Rab11. Rab11 identifies more and different exosome populations. With Rab11 staining, CD68 positive exosomes containing CD86 positive and negative exosomes are identified, and CD86 positive exosomes stained by Rab11 contain exosomes which are CD68 positive and negative.

The scale in FIGS. 10 to 14 denotes in dark negative, in light highly positive, and in grey dim positive.

### Example 11

C57BL/6 mice were injected 1×10⁸ pfu Adeno-GFP intravenously in the tail vene. 100 μl blood was drawn from these mice after 3 days of incubation. Plasma was prepared and incubated with anti-Rab11-PE antibody (dilution: 1/250) for 30 min at 21° C.

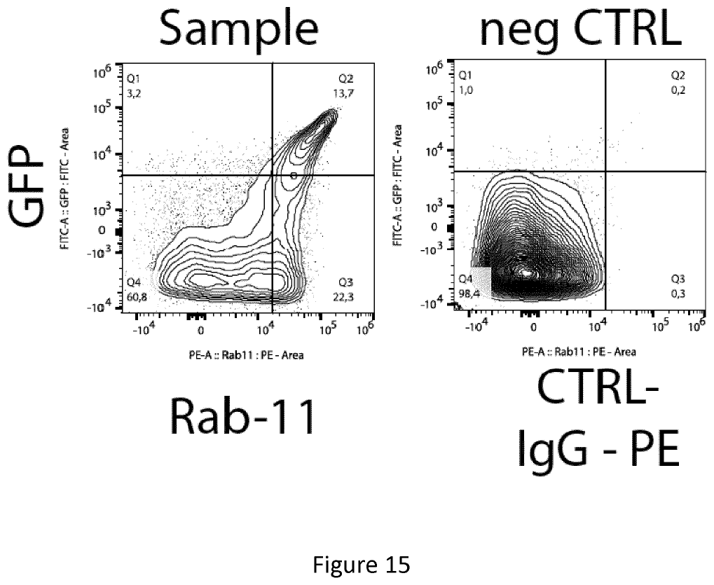

Samples were analyzed by FACS analysis (SP6800: threshold 0.1, Voltage 63). Results are shown in FIG. 15; left panel: Rab11 positive exosomes carrying GFP protein derived from the adeno-virus (virosomes); right panel control.

### Example 12

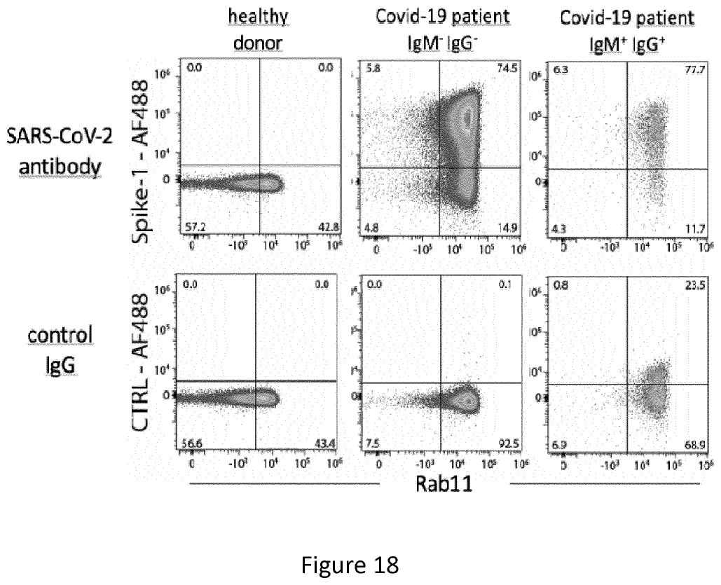

Rab11 positive exosomes carrying SARS-CoV-2 spike protein can be utilized for diagnosing a viral disease. The present inventors found that virus-infected cells release exosomes carrying virus protein on their surface. FIG. 18 shows detection of SARS-CoV-2 spike antigen on exosomes in blood of Covid-19 patients (right upper quadrant). Both Covid-19 patients were tested PCR positive. Control show the upper right quadrant empty. The fluorescence flow cytometry results demonstrate that Rab11-positive extracellular vesicles carry SARS-CoV-2 spike protein on their surface. Applying Rab11 mediated capturing of extracellular vesicles in blood from Covid-19 patients isolates exosomes containing SARS-CoV-2 protein.

### Example 13

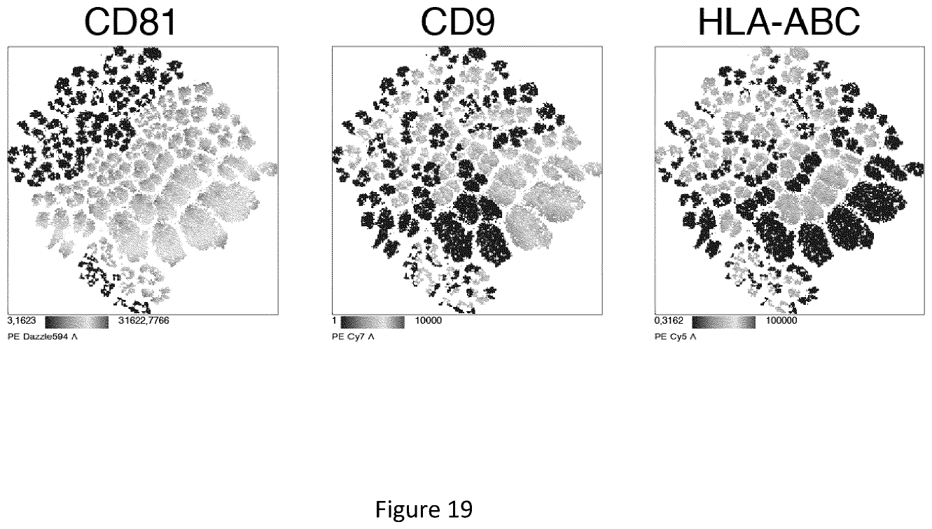

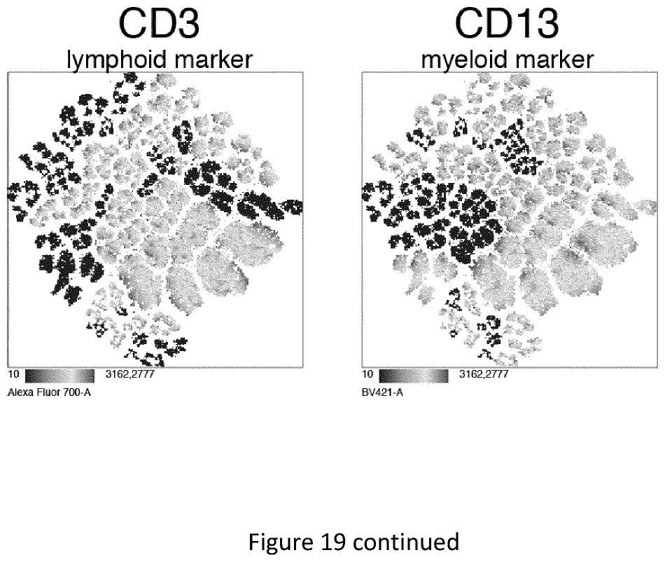

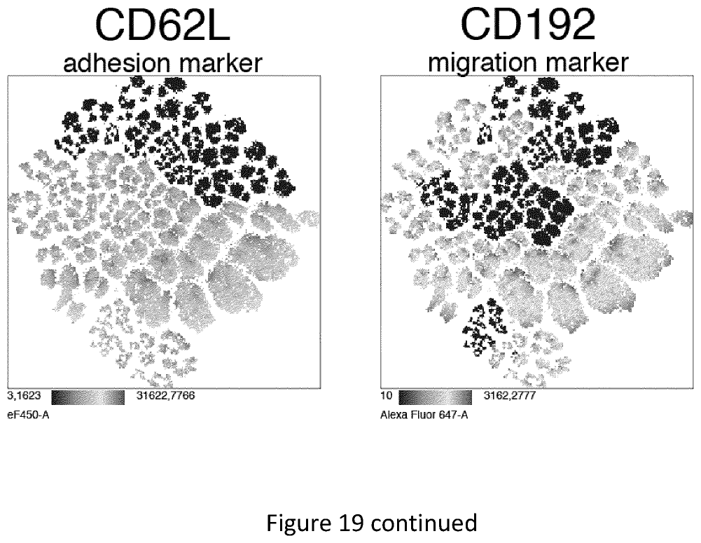

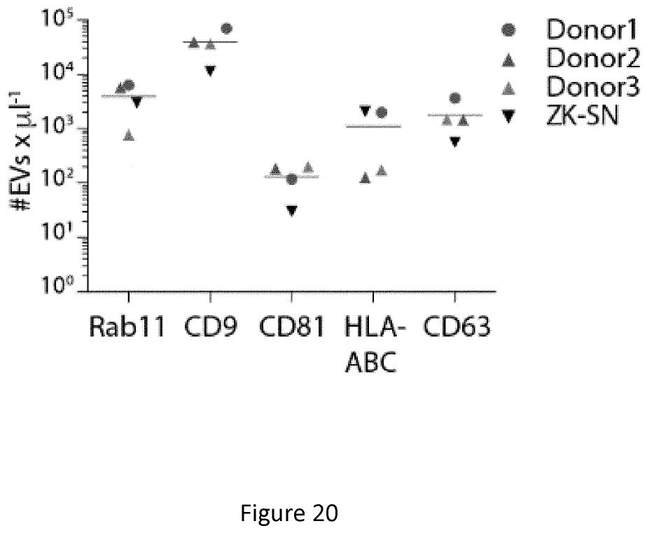

Phenotypic profiling of Rab11 positive exosomes. By applying stochastic neighborhood embedding (t-SNE), Rab11 positive exosomes were further separated into particles carrying markers defining immune cell lineages, i.e. T cells, B cells, myeloid cells, etc. Stochastic neighborhood analysis of the results obtained from the analysis of single EVs (flow cytometric results from phenotypic profiling of Rab11 positive exosomes from human plasma) showed distinct populations (FIG. 4) and revealed the power of this approach to characterize EVs based on a deep phenotypic profiling of the molecules expressed on their surface. Results are shown in FIG. 19.

### Example 14

For staining exosomes in the samples from plasma of healthy volunteers and supernatant of a mixture of breast cancer cell line (BT474, SKBR3, MCF7, OVCAR2, OVMZ6), 2×50 μl each were transferred into a 96-well plate and fluorescently labelled antibodies (Rab11, CD9, CD81, HLA-ABC, CD63) were added to one well each.

The samples were incubated for 30 min on ice in the dark before they were analyzed by flow-cytometry. The threshold value was set to 0.1% to make sure not to lose any exosomes and the sample flow rate was 1 to guarantee that only single exosomes pass the laser beam. Donor 1, 2, 3 denote plasma samples of three different healthy volunteers, ZK-SN denotes cell culture supernatant of breast cancer cell lines. The experiment shows that using Rab11 as marker yields the best results. CD9 does not only purify exosomes but also microvesicles derived from platelets and thus leads to higher amounts of total extracellular vesicles (EV) in the experiment.

### Items of the Invention

The present invention provides the following items:

Item 1: A method for producing an exosome enriched fraction from a sample, the method comprising the steps of:

i) contacting a first binding agent that specifically binds to the extra-vesicular part of Rab11 or a second binding agent that specifically binds to the extra-vesicular part of Rab4, or a combination thereof, with the sample under conditions allowing binding of said first binding agent and/or second binding agent to exosomes; and

ii) separating exosomes to which the first binding agent and/or the second binding agent is bound from the sample.

Item 2: The method of item 1, wherein:
a) the first binding agent comprises a first label and/or the second binding agent comprises a second label; or
b) the first binding agent and/or the second binding agent is bound by a third binding agent specifically binding to the first binding agent and/or the second binding agent, wherein the third binding agent comprises a third label; or
c) the first binding agent and/or second binding agent is covalently or non-covalently bound on a solid surface.

Item 3: The method of item 2, wherein the first, second and third label is independently selected from the group consisting of an enzyme label, a fluorescence label, a radioactive label, a magnetic label, a peptide or protein label, or a quantum dot.

Item 4: The method of any of items 1 to 3, wherein step ii) comprises the step of detecting the first and/or second antigen binding agent.

Item 5: The method of item 2 b) or 3, wherein step ii) comprises the step of detecting the third antigen binding agent.

Item 6: The method of any one of items 1 to 5, wherein the sample is a body fluid, preferably wherein the body fluid is selected from the group consisting of plasma, ascites, cerebral fluid, bone marrow, urine, faeces or bronco-alveolar washing.

Item 7: The method of any one of items 1 to 6, wherein the first binding agent binds to the extra-vesicular part of Rab11A and the second binding agent binds to the extra-vesicular part of Rab4A.

Item 8: The method of any one of items 1 to 7, wherein the method further comprises prior to step i):

a) suspending or solubilizing the sample; and
b) enriching exosomes from the sample based on size and/or density.

Item 9: The method of any one of items 1 to 8, wherein step ii) comprises flow cytometry, magnetic or microbead separation, or chromatography.

Item 10: A method for diagnosing cancer, comprising:
a) producing an exosome enriched fraction by a method of any one of items 1 to 9, and
b) detecting within the exosome enriched faction exosomes presenting a cancer antigen, preferably wherein the cancer antigen is GPER-1.

Item 11: The method according to item 10, wherein step b) comprises detecting the GPER-1 presenting exosomes with an anti-GPER-1 antibody.

Item 12: The method of item 10 or 11, wherein the cancer is breast cancer.

Item 13: A method for diagnosing a virus disease, comprising:
a) producing an exosome enriched fraction by a method of any one of items 1 to 9, and
b) detecting within the exosome enriched faction exosomes presenting a viral antigen.

Item 14: The method according to item 13, wherein the virus antigen is a virus surface protein, preferably a spike protein, most preferably a spike protein of the SARS-CoV-2 or Sars-CoV-1 virus.

Item 15: A method to quantify and/or qualify tumor-related exosomes in a sample, said method comprising the steps of:
a) producing an exosome enriched fraction by a method of any one of items 1 to 9; and
b) detecting tumor-related exosomes in the exosome enriched fraction of step a) with at least one binding agent specifically binding to a tumor antigen.

Item 16: The method of item 15, wherein the tumor antigen is selected from the group consisting of GPER-1, CD 247, and phosphatidylserine.

Item 17: A method for monitoring tumor growth, said method comprising the step of:

periodically quantifying the number of tumor related exosomes in a sample with the method according to item 15 or 16, wherein an increase in the number of tumor related exosomes between two quantifications indicates tumor growth.

Item 18: A method for monitoring a virus disease, said method comprising the step of:

periodically quantifying the number of virus related exosomes in a sample with the method according to any one of items 1 to 9 or according to item 13 or 14; optionally further comprising the step of detecting within the exosome enriched faction exosomes presenting a viral antigen, preferably wherein the viral antigen is a virus surface protein, more preferably a spike protein, most preferably a spike protein of the SARS-CoV-2 or Sars-CoV-1 virus.

Item 19: The method according to any one of the items 1 to 18, wherein the sample is obtained from a subject known or suspected to suffer from a disease, wherein the method further comprises comparing the quantity of exosomes in the sample of the subject known or suspected to suffer from a disease with the quantity of similar exosomes known to be present in a sample of a healthy subject, wherein an increase in the quantity of exosomes in the sample of the subject known or suspected to suffer from a disease is indicative of the presence or stage of the disease, preferably indicative of the presence or stage cancer.

Item 20: The method of item 19, wherein comparing the quantity of exosomes comprises applying CD mapping and t-SNE analysis.

Item 21: The method according to any one of the preceding items, further comprising analyzing surface markers and/or the content of the exosomes, preferably analyzing peptides, proteins, microRNA, DNA, and/or RNA.

Item 22: The method of item 21, wherein analyzing the content of the exosomes comprises DNA mutation analysis, RNA expression, DNA methylation quantification and/or protein expression.

Item 23: The method of any one of the preceding items, wherein the antigen binding agent is an antibody.

Item 24: A kit for performing the method according to any one of items 1 to 23, comprising:
(i) a first binding agent that specifically binds to the extra-vesicular part of Rab 11 or a second binding agent that specifically binds to the extra-vesicular part of Rab4, or a combination thereof; and
(ii) instructions for using the first and/or the second binding agent for binding of said first and/or second binding agent to exosomes in a sample.

Item 25: Method of diagnosing cancer or a virus disease in a subject, the method comprising the steps of:
a) obtaining a sample from a person known to or suspected of having cancer or suspected to have a viral infection;
b) producing an exosome enriched fraction from the sample, comprising:
   i) contacting a first binding agent that specifically binds to the extra-vesicular part of Rab11 or a second binding agent that specifically binds to the extra-vesicular part of Rab4, or a combination thereof, with the sample under conditions allowing binding of said first binding agent and/or second binding agent to exosomes; and
   ii) separating exosomes to which the first binding agent and/or the second binding agent is bound from the sample;
c) detecting tumor-related or virus-related exosomes in the exosome enriched fraction of step a) with at least one binding agent specifically binding to a tumor antigen or a virus antigen.

Item 26: The method of item 25, wherein
a) the first binding agent comprises a first label and/or the second binding agent comprises a second label; or
b) the first binding agent and/or the second binding agent is bound by a third binding agent specifically binding to the first binding agent and/or the second binding agent, wherein the third binding agent comprises a third label; or
c) the first binding agent and/or second binding agent is covalently or non-covalently bound on a solid surface.

Item 27: The method of item 26, wherein the first, second and third label is independently selected from the group consisting of an enzyme label, a fluorescence label, a radioactive label, a magnetic label, a peptide or protein label, or a quantum dot.

Item 28: The method of item 25, wherein the sample is a body fluid, preferably wherein the body fluid is selected from the group consisting of plasma, ascites, cerebral fluid, bone marrow, urine, faeces or bronco-alveolar washing.

Item 29: The method of item 25, wherein the first binding agent binds to the extra-vesicular part of Rab11A and the second binding agent binds to the extra-vesicular part of Rab4A.

Item 30: The method of item 25, wherein the method further comprises prior to step b)
a) suspending or solubilizing the sample; and
b) enriching exosomes from the sample based on size and/or density.

Item 31: The method of item 25, wherein step ii) comprises flow cytometry or chromatography.

Item 32: The method of item 25, wherein the tumor antigen is selected from the group consisting of GPER-1, CD247, or phophatidylserine.

Item 33: The method of item 31, wherein the antigen binding agent is an antibody.

### LITERATURE

Barroso M M. Quantum Dots in Cell Biology; J. Histochem. Cytochem., 2011. Vol. 59(3): 237-251
Böing A N, van der Pol E, Grootemaat A E, Coumans F A, Sturk A, Nieuwland R (2014). Single-step isolation of extracellular vesicles by size-exclusion chromatography. Journal of Extracellular Vesicles. 3: 23430
Blanc L and Vidal M. New insights into the function of Rab GTPases in the context of exosomeal secretion. Small GTPases 2018. Vol. 9, NOS 1-2, 95-106.
Brennetta J. Crenshaw, Linlin Gu, Brian Sims and Qiana L. Matthews1, Exosome Biogenesis and Biological Function in Response to Viral Infections. The Open Virology Journal DOI: 10.2174/1874357901812010134, 2018, 12, 134-148
Harding C, Heuser J, Stahl P. Receptor-mediated endocytosis of transferrin and recycling of the transferrin receptor in rat reticulocytes. J. Cell Biol. 1983; 97:329-339
Hench I B, Hench J, Tolnay M. Liquid Biopsy in Clinical Management of Breast, Lung, and Colorectal Cancer. Frontiers in Medicine 2018. Vol. 5, Article 9
Jia et al. Exosome: emerging biomarker in breast cancer. Oncotarget. 2017; 8:41717-41733
Li P, Kaslan M, Lee S H, Yao J and Gao Z. Progress in Exosome Isolation Techniques. Theranosics 2017. Vol. 7 (3): 789-804
Johnstone R M, Bianchini A, Teng K. Reticulocyte maturation and exosome release: transferrin receptor containing exosomes shows multiple plasma membrane functions. Blood 1989; 74: 1844-1851.
McAndrews K and Kallun R. Mechanisms associated with biogenesis of exosomes in cancer. Molecular Cancer 2019. DOI.org/10.1186/s12943-019-0963-9
Pan B T, Johnstone R M. Fate of the transferrin receptor during maturation of sheep reticulocytes in vitro: selective externalization of the receptor. Cell 1983; 33:967-978.
Savina A, Vidal M, Colombo M I. The exosome pathway in K562 cells is regulated by Rab11. J Cell Sci 2002; 115:2505-15; PMID:12045221[54] Vidal M, Mangeat P, Hoekstra D. Aggregation reroutes molecules from a recycling to a vesicle-mediated secretion pathway during reticulocyte maturation. J Cell Sci 1997; 110(16):1867-77; PMID:9296387
Tauro B J, Greening D W, Mathias R A, Ji H, Mathivanan S, Scott A M, Simpson R J (February 2012). “Comparison of ultracentrifugation, density gradient separation, and immunoaffinity capture methods for isolating human colon cancer cell line LIM1863-derived exosomes”. Methods. 56 (2): 293-304
Thind A, Wilson C (2016). “Exosomal miRNAs as cancer biomarkers and therapeutic targets”. Journal of Extracellular Vesicles. 5: 31292
van der Pol E, Boing A N, Harrison P, Sturk A, Nieuwland R (July 2012). “Classification, functions, and clinical relevance of extracellular vesicles”. Pharmacological Reviews. 64 (3): 676-705
Van Deun J, Mestdagh P, Sormunen R, Cocquyt V, Vermaelen K, Vandesompele J, Bracke M, De Wever O, Hendrix A (2014). “The impact of disparate isolation methods for extracellular vesicles on downstream RNA profiling”. Journal of Extracellular Vesicles. 3: 24858.

1. A method for producing an exosome enriched fraction from a sample, the method comprising the steps of:
i) contacting a first binding agent that specifically binds to the extra-vesicular part of Rab11 or a second binding agent that specifically binds to the extra-vesicular part of Rab4, or a combination thereof, with the sample under conditions allowing binding of said first binding agent and/or second binding agent to exosomes; and
ii) separating exosomes to which the first binding agent and/or the second binding agent is bound from the sample.

2. The method of claim 1, wherein:
a) the first binding agent comprises a first label and/or the second binding agent comprises a second label; or
b) the first binding agent and/or the second binding agent is bound by a third binding agent specifically binding to the first binding agent and/or the second binding agent, wherein the third binding agent comprises a third label; or
c) the first binding agent and/or second binding agent is covalently or non-covalently bound on a solid surface.

3. The method of claim 2, wherein the first, second and third label are independently selected from the group consisting of an enzyme label, a fluorescence label, a radioactive label, a magnetic label, a peptide or protein label, and a quantum dot.

4. The method of claim 1, wherein step ii) comprises the step of detecting the first and/or second antigen binding agent.

5. The method of claim 2 b), wherein step ii) comprises the step of detecting the third antigen binding agent.

6. The method of claim 1, wherein the sample is a body fluid, preferably wherein the body fluid is selected from the group consisting of plasma, ascites, cerebral fluid, bone marrow, urine, faeces or bronco-alveolar washing.

7. The method of claim 1, wherein the first binding agent binds to the extra-vesicular part of Rab11A and the second binding agent binds to the extra-vesicular part of Rab4A,
preferably wherein the method further comprises prior to step i):
a) suspending or solubilizing the sample; and
b) enriching exosomes from the sample based on size and/or density,
more preferably wherein step ii) comprises flow cytometry, magnetic or microbead separation, or chromatography.

8. A method for diagnosing cancer, comprising:
a) producing an exosome enriched fraction by the method of claim 1, and
b) detecting within the exosome enriched faction exosomes presenting a cancer antigen, preferably wherein the cancer antigen is GPER-1.

9. The method according to claim 8,
wherein step b) comprises detecting the GPER-1 presenting exosomes with an anti-GPER-1 antibody and/or
wherein the cancer is breast cancer.

10. A method for detecting or diagnosing a virus disease, comprising:
a) producing an exosome enriched fraction by a method of claim 1, and
b) detecting within the exosome enriched faction exosomes presenting a viral antigen,
preferably wherein the viral antigen is a virus surface protein, more preferably a spike protein, most preferably a spike protein of the SARS-CoV-2 or Sars-CoV-1 virus.

11. A method to quantify and/or qualify tumor-related exosomes in a sample, said method comprising the steps of:
a) producing an exosome enriched fraction by the method of claim 1; and
b) detecting tumor-related exosomes in the exosome enriched fraction of step a) with at least one binding agent specifically binding to a tumor antigen,
preferably wherein the tumor antigen is selected from the group consisting of GPER-1, CD247, and phosphatidylserine.

12. The method of claim 11, further comprising periodically quantifying the number of tumor related exosomes in the sample,
wherein an increase in the number of tumor related exosomes between two quantifications indicates tumor growth.

13. The method of claim 10, further comprising:
periodically quantifying the number of virus related exosomes in the sample to monitor the virus disease, and
optionally further comprising the step of detecting within the exosome enriched faction exosomes presenting a viral antigen, preferably wherein the viral antigen is a virus surface protein, more preferably a spike protein, most preferably a spike protein of the SARS-CoV-2 or Sars-CoV-1 virus.

14. The method according to claim 1,
wherein the sample is obtained from a subject known or suspected to suffer from a disease, wherein the method further comprises comparing the quantity of exosomes in the sample of the subject known or suspected to suffer from a disease with the quantity of similar exosomes known to be present in a sample of a healthy subject, wherein an increase in the quantity of exosomes in the sample of the subject known or suspected to suffer from a disease is indicative of the presence or stage of the disease, preferably indicative of the presence or stage cancer, preferably wherein comparing the quantity of exosomes comprises applying CD mapping and t-SNE analysis, and/or
further comprising analyzing surface markers and/or the content of the exosomes, preferably analyzing peptides, proteins, microRNA, DNA, and/or RNA, preferably wherein analyzing the content of the exosomes comprises DNA mutation analysis, RNA expression, DNA methylation quantification and/or protein expression, and/or
wherein the antigen binding agent is an antibody.

15. A kit, comprising:
a first binding agent that specifically binds to the extra-vesicular part of Rab11 or a second binding agent that specifically binds to the extra-vesicular part of Rab4, or a combination thereof; and
(ii) instructions for using the first and/or the second binding agent for binding of said first and/or second binding agent to exosomes in a sample.
